(19) United States
(12) Patent Application Publication
(10) Pub. No.: US 2025/0359914 A1
(43) Pub. Date: Nov. 27, 2025

(54) DISTAL TIP STRUCTURE FOR CRYOABLATION PROBE

(71) Applicant: Boston Scientific Scimed, Inc., Maple Grove, MN (US)

(72) Inventors: Kyle True, Minneapolis, MN (US); Cory Ross Stenberg, St. Michael, MN (US); David Cory Kirt, Minnetonka, MN (US); Brandon Eilers, Brooklyn Park, MN (US); Zachary Nickle, Minneapolis, MN (US)

(21) Appl. No.: 18/671,742

(22) Filed: May 22, 2024

(51) Int. Cl. A61B 18/02; A61B 18/00

(52) U.S. Cl. CPC A61B 18/02; A61B 2018/00077; A61B 2018/00101; A61B 2018/00172; A61B 2018/00577; A61B 2018/0212; A61B 2018/0262

(57) ABSTRACT

In an embodiment, a cryoablation probe includes a shaft, where the shaft has a supply tube, a return tube surrounding the supply tube, an insulated portion, wherein a vacuum circuit runs through the insulated portion between the return tube and an insulating shaft, and an expansion chamber extending distally to the insulated portion. The probe further includes a distal tip configured to seal a distal end of the return tube. The distal tip has a tip portion extending distally from the distal end of the return tube and a plug portion configured to be inserted inside of the return tube. The probe includes a compressive element surrounding the return tube and the plug portion of the distal tip, configured to seal the distal tip to the return tube.

### TECHNICAL FIELD

Embodiments herein relate to cryoablation systems and more particularly to distal tip structures for cryoablation systems.

### BACKGROUND OF THE INVENTION

During cryosurgery, a surgeon may deploy one or more cryoprobes to ablate a target area of a patient anatomy by freezing and thawing the tissue. In one example, a cryoprobe uses the Joule-Thomson effect to produce cooling or heating of the probe tip. In such cases, the expansion of a cryofluid in the cryoablation probe from a higher pressure to a lower pressure leads to cooling of the device tip to temperatures at or below those corresponding to cryoablation a tissue in the vicinity of the tip. Heat transfer between the expanded cryofluid and the outer walls of the cryoprobe leads to formation of an ice ball, in the tissue around the tip and consequent cryoablation of the tissue.

### SUMMARY OF THE INVENTION

In a first aspect, a cryoablation probe includes a working fluid circuit, a vacuum circuit, and a shaft. The shaft includes a supply tube, a return tube surrounding the supply tube, an insulated portion, wherein the vacuum circuit runs through the insulated portion between the return tube and an insulating shaft, and an expansion chamber extending distally to the insulated portion, wherein fluid from the working fluid circuit travels through the supply tube and expands in the expansion chamber. The probe further includes a distal tip configured to seal a distal end of the return tube. The distal tip includes a tip portion extending distally from the distal end of the return tube and a plug portion configured to be inserted inside of the return tube. The probe further includes a compressive element surrounding the return tube and the plug portion of the distal tip, configured to seal the distal tip to the return tube.

In a second aspect, in addition to one or more of the preceding or following aspects, or in the alternative to some aspects, at least a portion of the plug portion can have a first portion with a diameter that is larger than an inner diameter of the return tube and the compressive element can be a ring.

In a third aspect, in addition to one or more of the preceding or following aspects, or in the alternative to some aspects, the plug portion includes a neck section having a minimum neck diameter and a base section having a minimum base diameter, wherein the base section can be located proximally to the neck section along the return tube, wherein the minimum neck diameter is smaller than the inner diameter of the return tube, and wherein the base section includes the first portion having a diameter larger than the inner diameter of the return tube.

In a fourth aspect, in addition to one or more of the preceding or following aspects, or in the alternative to some aspects, the plug portion includes a first shoulder between the neck section and the base section.

In a fifth aspect, in addition to one or more of the preceding or following aspects, or in the alternative to some aspects, the neck section includes a protrusion configured to form a seal with the return tube, wherein the ring surrounds the protrusion.

In a sixth aspect, in addition to one or more of the preceding or following aspects, or in the alternative to some aspects, the base section includes a barb configured to form an interference fit with the return tube, wherein the first portion of the plug portion can be the barb.

In a seventh aspect, in addition to one or more of the preceding or following aspects, or in the alternative to some aspects, a proximal end of the tip portion can have a larger diameter than a distal end of the plug portion, and wherein the distal tip includes a tip shoulder between the tip portion and the plug portion, wherein a distal end of the return tube abuts the distal tip at the tip shoulder.

In an eighth aspect, in addition to one or more of the preceding or following aspects, or in the alternative to some aspects, a distal end of the compressive element surrounds the distal tip at the tip shoulder or distally to the tip shoulder.

In a ninth aspect, in addition to one or more of the preceding or following aspects, or in the alternative to some aspects, the ring includes a swage ring.

In a tenth aspect, in addition to one or more of the preceding or following aspects, or in the alternative to some aspects, the ring includes a metal ring.

In an eleventh aspect, in addition to one or more of the preceding or following aspects, or in the alternative to some aspects, the compressive element includes a compressive wrap surrounding the return tube and the plug portion of the distal tip, configured to seal the distal tip to the return tube.

In a twelfth aspect, in addition to one or more of the preceding or following aspects, or in the alternative to some aspects, the plug portion includes a neck section having a neck diameter and a base section having a base diameter, wherein the base section can be located proximally to the neck section along the return tube, wherein the neck diameter can be smaller than an inner diameter of the return tube, and wherein the base diameter can be equal to or larger than the inner diameter of the return tube.

In a thirteenth aspect, in addition to one or more of the preceding or following aspects, or in the alternative to some aspects, the plug portion includes a ramp between the neck section and the base section, wherein a diameter of the plug portion increases over a length of the ramp moving in a proximal direction from the neck section to the base section.

In a fourteenth aspect, in addition to one or more of the preceding or following aspects, or in the alternative to some aspects, the compressive wrap includes a filament wrapped around the return tube and plug portion of the distal tip with a wrap threshold tension.

In a fifteenth aspect, in addition to one or more of the preceding or following aspects, or in the alternative to some aspects, the probe includes an adhesive configured to encapsulate the compressive wrap, wherein the adhesive can be configured to maintain the wrap threshold tension of the filament.

In a sixteenth aspect, in addition to one or more of the preceding or following aspects, or in the alternative to some aspects, the tip portion includes an atraumatic surface.

In a seventeenth aspect, in addition to one or more of the preceding or following aspects, or in the alternative to some aspects, a proximal end of the distal tip includes one or more curved edges.

In an eighteenth aspect, a method of making a shaft component of a cryoablation probe includes providing a tube component of a cryoablation expansion chamber and providing a distal tip configured to seal a distal end of the tube component, the distal tip including a tip portion configured to extend distally from the distal end of the tube component and a plug portion configured to be inserted inside of the tube. The method further includes inserting the distal tip plug portion into a distal end of the tube component so that the plug portion can be within the tube component and the tip portion extends distally from the distal end of the tube component. The method further includes applying a compressive force to a compressive element surrounding the tube component and the plug portion of the distal tip, wherein the compressive element seals the distal tip to the tube component, and positioning a supply tube within the tube component, wherein the supply tube can be configured to supply a fluid of a working fluid circuit, wherein the fluid expands in the expansion chamber.

In a nineteenth aspect, in addition to one or more of the preceding or following aspects, or in the alternative to some aspects, the compressive element is a ring and the step of applying the compressive force includes positioning the ring surrounding the tube component and the plug portion of the distal tip, and compressing the ring, wherein at least a first portion of the plug portion has a diameter that is equal to or larger than an inner diameter of the tube component.

In a twentieth aspect, in addition to one or more of the preceding or following aspects, or in the alternative to some aspects, the compressive element includes a compressive wrap and wherein applying the compressive force includes wrapping the compressive wrap around the tube component and the plug portion of the distal tip.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cryoablation system in accordance with various embodiments herein.

FIG. 2 is a schematic view of portions of a cryoablation system in accordance with various embodiments herein.

FIG. 3 is a schematic view of a portion of a cryoablation shaft shown in accordance with various embodiments herein.

FIG. 4 is a cross sectional view of the shaft of FIG. 3 taken along section 4-4 in FIG. 3 in accordance with various embodiments herein.

FIG. 5 is a cross-sectional view of the shaft of FIG. 3 taken along section 5-5 in FIG. 3 in accordance with various embodiments herein.

FIG. 6 is a schematic view of the biliary system in accordance with various embodiments herein.

FIG. 7 is a schematic view of the growth of an ice ball generated by a cryoablation system in accordance with various embodiments herein.

FIG. 8-13 are schematic views of a portion of a cryoablation shaft in accordance with various embodiments herein.

FIG. 14 is a schematic view of a distal tip in accordance with various embodiments herein.

FIG. 15 is an exploded view of a distal tip and the distal end of a cryoablation shaft in accordance with various embodiments herein.

FIG. 16 is a schematic view of a distal end of a cryoablation shaft sealed with a distal tip in accordance with various embodiments herein.

FIG. 17 is schematic view of a distal tip in accordance with various embodiments herein.

FIG. 18 is a schematic view of a distal tip in accordance with various embodiments herein.

While embodiments are susceptible to various modifications and alternative forms, specifics thereof have been shown by way of example and drawings and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Some cryoablation systems may be useful for ablating lesions in the biliary system or other difficult to access portions of the human anatomy. In such cases, the cryoprobes may have to navigate tortuous passageways. Cryoprobes with rigid shafts may not be suitable for such applications.

The present disclosure is directed towards distal tip structures for cryoablation systems. During cryosurgery, a surgeon may deploy one or more cryoprobes to ablate a target area of a patient anatomy by freezing and thawing the tissue. In a cryoprobe, a rigid distal tip can be joined to the distal end of the cryoablation shaft using a compressive element such as a swage ring or a fibrous compressive wrap. The distal tip can include one or more mechanical features that facilitate the seal between the distal tip and cryoablation shaft, enhancing the robustness of the cryoprobe and reducing the instances of fluid leakage. Examples of mechanical features and compressive elements described herein work together to provide the seal.

The concepts described herein can be applied in the context of the cryoablation systems described in US Published Patent Application US2021/00045793, titled “Dual Stage Cryocooler,” and US Published Patent Application US2021/00045794, titled “Flexible Cryoprobe,” both filed Aug. 14, 2020, and both incorporated herein by reference in their entireties.

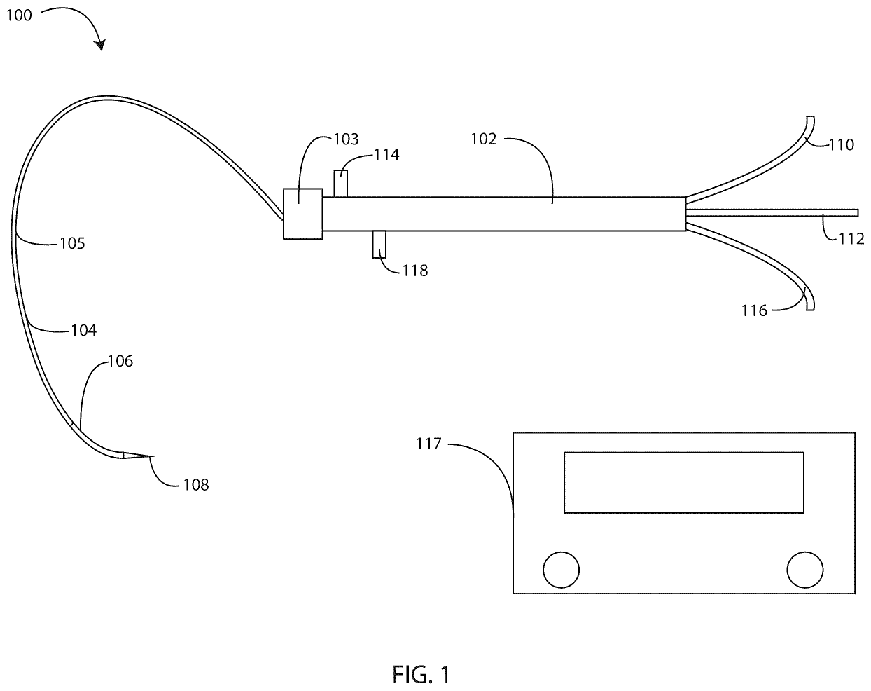

Referring now to FIG. 1, a schematic view of a cryoablation system is shown in accordance with various embodiments herein. In various embodiments, the cryoablation system can include a handle 102 and a shaft 104. In various embodiments, the shaft 104 is insertable into the handle 102 and can be securely attached to the handle with shaft-handle connector 103. In various embodiments, the shaft 104 and the shaft-handle connector 103 of a cryoablation system 100 can form a catheter assembly. In some embodiments, the catheter assembly includes the components of the cryoablation system that are to be replaced each time a cryoablation procedure is performed. In some aspects, the cryoablation system 100 may include a working fluid source 110, a pre-cooler fluid source 112, and vacuum source 114 which are each connectible to the cryoablation system 100.

The three sources correspond to three independent circuits in the cryoablation system 100: pre-cooler, working fluid, and active vacuum. In some embodiments, the working fluid source 110 and pre-cooler fluid source 112 connect to the base of the handle 102 of the cryoablation system 100 and vacuum source 114 connects near the distal end of the handle, adjacent to the shaft-handle connector 103. The cryoablation system may further include a pre-cooler gas exhaust 116 and a working gas exhaust 118 connecting to the handle 102. In various embodiments, the shaft-handle connector 103 functions as a manifold to ensure each of the flow circuits remain isolated from one another.

In some embodiments, the cryoablation system 100 includes a console 117. The console may be used to control the system and may be in electrical and fluid communication with the handle and cryoablation assembly. In some embodiments, the working fluid source 110, pre-cooler fluid source 112, vacuum source 114 may each be connectable to a console 117 of the cryoablation system 100 using conduits. In one embodiment, the pre-cooler gas exhaust 116, working gas exhaust 118, or both can connect to a conduit which carries the exhaust back to the console 117 or other location in the procedure room where the exhaust is vented to the ambient environment at an appropriate location. It should be noted that various sources and exhausts may be placed in position and in any suitable configuration along the handle 102, and that the arrangement of FIG. 1 is just one example of a suitable configuration.

An example of specifications and functions of each of these circuits is provided in the following paragraph. However, it should be noted that the particular fluids and pressure values are meant for exemplary purposes and other configurations are possible. In an embodiment, the pre-cooler circuit can contain 24.1 mega Pascals (MPa) pressurized Argon. The precooler circuit can cool the incoming stream of working fluid and can operate in the handle. In an embodiment, the working fluid circuit can contain 12.4 MPa pressurized Argon and/or 12.4 MPa pressurized Helium. The working fluid circuit generates and/or thaws ice balls. The working fluid circuit can operate in the handle, the insulated portion or insulated zone of the shaft, and the expansion chamber of the shaft. In an embodiment, the active vacuum can hold a vacuum of less than or equal to 6.67 Pascals (Pa). The active vacuum can insulate the shaft. The active vacuum can operate in the handle and the insulated zone of the shaft.

In various embodiments, the working fluid circuit runs through both the handle 102 and the shaft 104 of the cryoablation system 100 and carries the fluid which both generates and thaws the ice ball. The term “fluid circuit” is used throughout the application, and could be replaced with gas circuit, liquid circuit, fluid chamber, gas chamber, or liquid chamber in various embodiments. The term “fluid” is used throughout and could be replaced with gas or liquid in various embodiments. During the ablation (freeze cycle), 12.4 MPa argon is circulated through the probe to generate the ice ball in the patient's body surrounding the expansion chamber 106. The working fluid can be any suitable cooling fluid (e.g., nitrogen, air, argon, krypton, xenon, N2O, CO2, CF4). In some embodiments, the pressure of the high-pressure stream of the working fluid can be greater than or equal to 6.9 MPa, 8.3 MPa, 9.7 MPa, 11.0 MPa, 12.4 MPa, 17.2 MPa, 27.6 MPa, or 41.4 MPa. In some embodiments, the pressure of the high-pressure stream of the working fluid can be less than or equal to 55.2 MPa, 34.5 MPa, 20.7 MPa, 18.6 MPa, 16.5 MPa, 14.5 MPa, or 12.4 MPa. In some embodiments, the pressure of the high-pressure stream of the working fluid can fall within a range of 6.9 MPa to 41.4 MPa, or 8.3 MPa to 27.6 MPa, or 9.7 MPa to 16.5 MPa, or 11.0 MPa to 14.5 MPa, or can be about 12.4 MPa. Accordingly, in the embodiments where the working fluid is a cooling fluid, the temperature of the working fluid at the expansion chamber 106 can be about 190 Kelvin. In some embodiments, the temperature of the working fluid can be less than or equal to 250 Kelvin, 200 Kelvin, 150 Kelvin, or 100 Kelvin, or can be an amount falling within a range between any of the foregoing.

In various embodiments, the pre-cooler circuit is fully contained within the handle 102. In various embodiments, the pre-cooler circuit is located in a console 117 of the system. In various embodiments, the pre-cooler circuit is located in a part of the catheter just proximal to the handle. In various embodiments, the pre-cooler circuit is located in a part of the catheter just distal to the handle. The pre-cooler circuit operates using argon or any other suitable cooling fluid in various embodiments. In some embodiments, the high-pressure stream of the pre-cooler fluid may be at a pressure greater than the pressure of the high-pressure stream of the working fluid. The pre-cooler fluid may, for instance, be supplied at pressures greater than about 13.8 MPa. In some embodiments, the pressure of the pre-cooler fluid can be greater than or equal to 10.3 MPa, 13.8 MPa, 17.2 MPa, 20.7 MPa, or 24.1 MPa. In some embodiments, the pressure of the pre-cooler fluid can be less than or equal to 31.0 MPa, 29.3 MPa, 25.9 MPa, or 24.1 MPa. In some embodiments, the pressure of the pre-cooler fluid can fall within a range of 10.3 MPa to 31.0 MPa, or 13.8 MPa to 29.3 MPa, or 17.2 MPa to 27.6 MPa, or 20.7 MPa to 25.9 MPa, or can be about 24.1 MPa.

In some embodiments, the outer surface of the shaft 104 may be thermally insulated from the inner surface of the shaft. In various embodiments, the vacuum circuit or vacuum chamber runs through both the handle 102 and the insulated zone 105 of the shaft 104. Vacuum is actively pulled along the insulated zone 105 of the shaft 104 throughout the cryoablation procedure, providing a protective barrier between the outer surface of the shaft 104 and the patient. In alternative embodiments, shaft insulation can be obtained by circulating fluid, gas, or a heated fluid throughout the shaft or by electrically heating portions of the shaft. In alternative embodiments, shaft insulation can be obtained by containing a non-circulating fluid or gas within an insulating shaft.

The shaft 104 can be of any suitable length capable of reaching the target anatomy in the subject. In some embodiments, the shaft length can be greater than or equal to 20 cm, 38 cm, 55 cm, 72 cm, or 90 cm. In some embodiments, the shaft length can be less than or equal to 150 cm, 135 cm, 120 cm, 105 cm, or 90 cm. In some embodiments, the shaft length can fall within a range of 20 cm to 150 cm, or 38 cm to 135 cm, or 55 cm to 120 cm, or 72 cm to 105 cm, or can be about 90 cm.

In various embodiments, certain portions of the shaft 104 may be flexible. In an embodiment, the entire length of the shaft may be flexible. For instance, the shaft may be bendable about its lengthwise axis. In some such embodiments, the shaft may have a shaft diameter configured such that the shaft may be sufficiently flexible to form a curve having a desired radius of curvature. For instance, the shaft may be sufficiently flexible, such that the shaft may form a curve having the smallest radius of curvature of less than or equal to 30 mm, 20 mm, 10 mm, or 5 mm.

In various embodiments, shaft 104 may include an insulated zone 105 and an expansion chamber 106. The insulated zone 105 defines the portion of shaft 104 that is insulated by the vacuum chamber. The expansion chamber 106 defines the portion of the shaft 104 that is not insulated by the vacuum and where the ice ball is generated. In various embodiments, shaft carries high pressure working fluid from the handle 102 to the expansion chamber 106, where it undergoes a Joule-Thompson expansion and corresponding temperature change. The working fluid exits down the shaft, through the handle, before venting to the atmosphere from the console, or into the handle and venting from the handle.

The distal end of the shaft may terminate in a distal operating tip 108. During use, the distal operating tip 108 is deployed in the body of a patient, is surrounded by tissue, and cryogenically ablates the tissue in some instances. The distal operating tip 108 may be advantageously configured to pierce tissue in some instances. For example, the distal operating tip 108 may include a sharp tip, such as a trocar tip. Alternatively, the distal operating tip 108 may not be a sharp tip. In some embodiments, the distal operating tip 108 can be an atraumatic tip designed to cause minimal tissue injury. In some embodiments, the distal operating tip 108 may also contain a working port configured for any of aspiration, delivery of therapeutics, and delivery of other devices including, but not limited to guide wires, imaging catheters, sensing devices, biopsy devices, balloons, and stents.

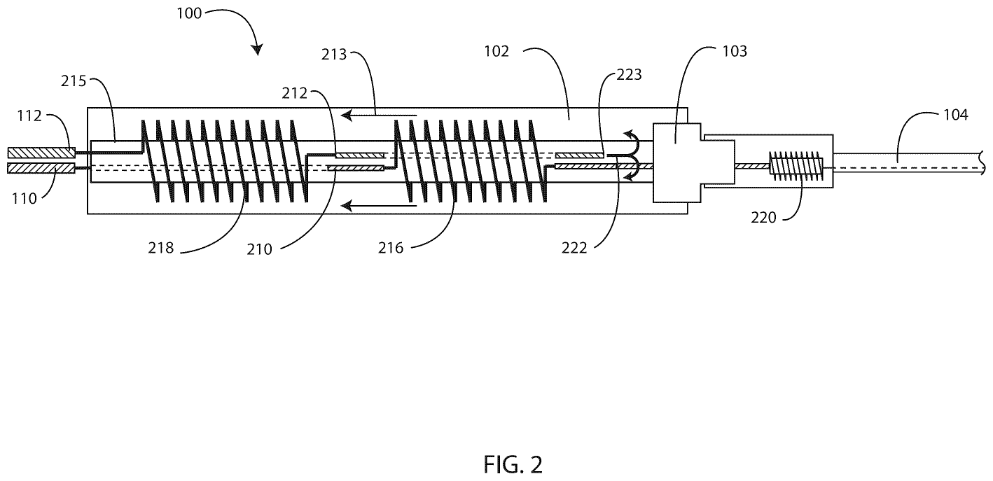

Handle with Pre-Cooler Circuit (FIG. 2)

Referring now to FIG. 2, a schematic view of portions of a cryoablation system is shown in accordance with various embodiments herein. In some aspects, the cryoablation system 100 may include a working fluid source 110 connecting to a working fluid circuit and a pre-cooler fluid source 112 connecting to a pre-cooler fluid circuit. The working fluid circuit may include a working fluid supply conduit 210 for carrying a high-pressure stream of the working fluid from the working fluid source 110 to the distal end of the shaft 104 (not shown in this view). The working fluid circuit may also include a working fluid return conduit (not shown in this view) for carrying a low-pressure stream of the working fluid from the distal end of the shaft back to the base of the handle 102.

The pre-cooler fluid circuit may include a pre-cooler supply circuit 212, which terminates at pre-cooler Joule-Thomson orifice 223 and carries a high-pressure stream of a pre-cooler fluid from the pre-cooler fluid source 112 to the pre-cooler fluid expansion region 222 in the handle 102. The pre-cooler fluid circuit may also include a pre-cooler return conduit (marked by arrows 213). The pre-cooler return conduit may be configured to carry the pre-cooler fluid away from the pre-cooler fluid expansion region 222 back to the base of the handle 102. The pre-cooler return conduit may be housed along with the pre-cooler supply circuit 212 and extend back to a control console and gas manifold.

In various embodiments, the pre-cooler fluid circuit may facilitate heat exchange between the working fluid and the pre-cooler fluid. For instance, the pre-cooler fluid circuit can be used to precool the high-pressure stream of the working fluid in embodiments where the working fluid cools upon expansion to cryogenically ablate tissue surrounding the distal operating tip 108. In various embodiments, the working fluid supply conduit 210 may include a first heat exchanger 216. The first heat exchanger 216 may facilitate heat exchange between the high-pressure stream of the working fluid in the working fluid supply conduit 210 and the low-pressure stream of the pre-cooler fluid in the pre-cooler return conduit.

In various embodiments, the pre-cooler supply conduit 212 may include a second heat exchanger 218 that permits heat exchange between the high-pressure stream of the pre-cooler fluid and the low-pressure stream of the pre-cooler fluid (e.g., recuperative heat exchange). In various embodiments aspects, the pre-cooler fluid may also be a cooling fluid. In such embodiments, recuperative heat exchange between the high-pressure stream of the pre-cooler fluid and the low-pressure stream of the pre-cooler fluid may remove heat from the high-pressure stream of the pre-cooler fluid. Accordingly, the second heat exchanger 218 may facilitate precooling the high-pressure stream of the pre-cooler fluid.

In various embodiments, the high-pressure stream of the pre-cooler fluid leaving the second heat exchanger 218 continues to flow through the pre-cooler supply conduit 212 to the pre-cooler fluid expansion region 222. In the pre-cooler fluid expansion region, which is fully contained in handle 102, the pre-cooler supply conduit 212 terminates in a Joule-Thomson orifice. The high-pressure stream of the pre-cooler fluid may undergo expansion at or downstream of the Joule-Thomson orifice in the pre-cooler fluid expansion region 222. The rapid drop in pressure causes a corresponding drop in temperature. The pre-cooler fluid expansion region 222 may be in fluid communication with the pre-cooler return conduit to carry the expanded low-pressure stream of the pre-cooler fluid (e.g., to vent to atmosphere, if the pre-cooler fluid circuit is an open circuit, or back to a pre-cooler fluid source if the pre-cooler fluid circuit is a closed circuit). After expansion at the Joule-Thomson orifice, the chilled pre-cooler fluid passes back through handle 102, in the annular space between the core tube 215 and the outer surface of the handle 102. As the pre-cooler fluid passes through the pre-cooler return conduit, it cools the working fluid at the first heat exchanger 216.

The working fluid circuit 210 may also include a third heat exchanger 220 in the shaft 104 of the cryoablation system that is configured for heat exchange (e.g., recuperative heat exchange) between the high-pressure stream of the working fluid in the working fluid supply circuit 210 and the low-pressure stream of the working fluid returning through the shaft 104 (not shown in this view).

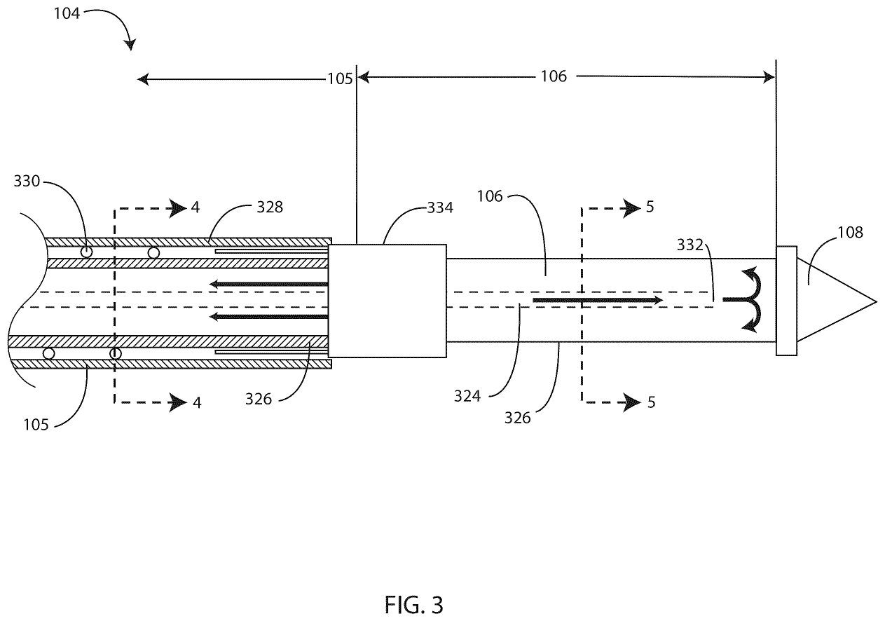

### Distal Tip and Expansion Chamber Details (FIG. 3)

Referring now to FIG. 3, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 which is located within a return tube 326, which is located within an insulating shaft 328. The concentric-shaft construction is designed to isolate the working fluid circuit 210 and vacuum chamber 336 from each other.

In various embodiments, after exiting the handle 102, the high-pressure flow of the working fluid travels down the supply tube 324. When the working fluid reaches the working fluid expansion chamber 106, the supply tube 324 terminates in a Joule-Thomson orifice 332 or distal outlet 332. The high-pressure stream of the working fluid may undergo expansion at or downstream of the Joule-Thomson orifice 332 in expansion chamber 106. The rapid drop in pressure causes a corresponding drop in temperature. Heat transfer between the expanded working and the outer walls of expansion chamber 106 leads to formation of an ice ball in the tissue around the tip 108 resulting in cryoablation of the tissue.

The expansion chamber 106 may be in fluid communication with the working fluid return conduit (defined by the annular space between the supply tube 324 and the inner surface of the return tube 326 of the expansion chamber) to carry the expanded low pressure stream of the working fluid (e.g., to vent to atmosphere, if the working fluid circuit is an open circuit, or back to a working fluid source if the working fluid circuit is a closed circuit). As the working fluid passes through the working fluid return conduit, it cools the working fluid input stream at the third heat exchanger 220 (FIG. 2).

In various embodiments, the working fluid is a cooling fluid and a cooling gas (e.g., nitrogen, air, argon, krypton, xenon, N₂O, CO₂, CF₄). In such cases, the high-pressure stream of the working fluid may be at a pressure such that expansion via the Joule-Thomson orifice 332 may result in the working fluid cooling to temperatures for cryogenically ablating tissue surrounding the expansion chamber 106. In certain aspects, the pressure of the high-pressure stream of the working fluid upstream of the Joule-Thomson orifice 332 can be between about 6.9 MPa and about 13.8 MPa (e.g., about 12.4 MPa). Accordingly, in the embodiments where the working fluid is a cooling fluid, the temperature of the working fluid after expansion from the Joule-Thomson orifice 332 can be greater than or equal to 150, 160, 170, 180, 190, or 200 Kelvin, or can be an amount falling within a range between any of the foregoing.

Cryoablation system 100 can be designed such that the outermost surface of the shaft does not cause thermal damage to non-target structures. In various embodiments, Ice ball formation is limited to the expansion chamber 106 of the shaft 104, which can also be referred to as the active region of the device. Selective ice ball formation is achieved by pulling a vacuum through the insulated zone 105 of shaft 104. In various embodiments, the cryoablation system 100 may be configured for establishing vacuum communication between the shaft 104 and vacuum source 114.

Referring back to FIG. 1, cryoablation system 100 may be configured to connect to a vacuum source 114 at handle 102. In various embodiments, the vacuum source 114 is configured to pull vacuum along the length of the insulated zone 105 of shaft 104. In an embodiment, vacuum is pulled between the outer diameter of the return tube 326 and the inner diameter of the insulating shaft 328 throughout the insulated zone 105 of shaft 104.

In various embodiments, the vacuum source 114 is configured to pull a vacuum within at least a portion of the handle 102. Such a configuration can insulate the handle 102 and protect the cryoablation system operator from cryogenic exhaust gases. In some embodiments the vacuum source 114 is connected to the handle 102 and the shaft 104 is in fluid communication with the handle 102 such that pulling a vacuum in the handle can also evacuate the space between the supply tube 324 and return tube 326. In other embodiments, the vacuum source 114 is connected directly to the shaft 104, for example, with the use of a t-fitting along the length of the shaft 104.

To provide for thermal insulation along the insulated zone 105 of shaft 104, the wall of the shaft is a double wall (a return tube surrounded by an insulating shaft) with a small gap between the return tube 326 and the insulating shaft 328. By pulling a vacuum between the return tube and the insulating shaft, convective heat transfer is prevented, so that the temperature of the working fluid does not ablate or cause uncontrolled apoptosis/necrosis to healthy non-target patient tissue along the insulated zone of the shaft. Adequate thermal insulation is obtained by actively pumping out the air in the gap and maintaining a vacuum of about 0.05 torr. However, other vacuum pressures may be appropriate depending on the configuration of the cryoablation system. In some embodiments, a supporting filament 330 is wrapped around the outer diameter of the return tube 326. One option for the filament material is a polymer such as polyether ether ketone (PEEK). The filament may prevent direct contact between the return tube outer surface and insulating shaft inner surface. Filament 330 minimizes thermal conduction between the inner and insulating shafts. Other alternatives may be used in place of the filament 330 such as an extruded tubing/co-extrusion shape or other features placed onto the shaft.

In some embodiments, the shaft may not include a filament. In such embodiments the return tube 326 and the insulating shaft 328 are selected to have material properties that are sufficient to minimize thermal conduction between the inner shaft and the insulating shaft.

A joint 334 is present at the junction of the insulated zone 105 and the expansion chamber 106. This joint is capable of sealing the vacuum layer.

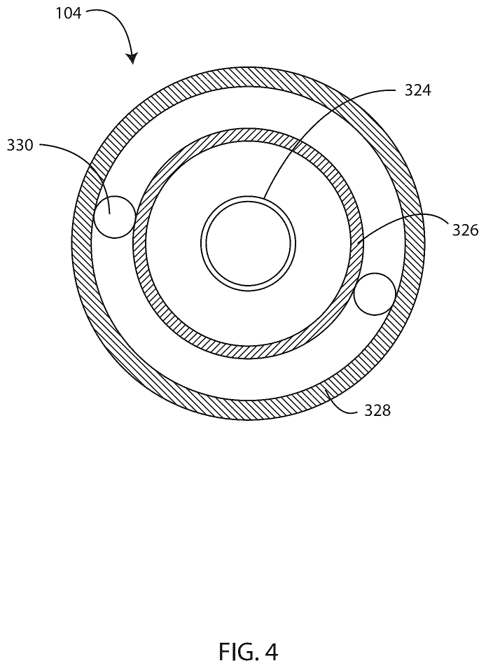

### Flexible Shaft Cross-Section, Dimensions and Materials (FIG. 4)

Referring now to FIG. 4, a cross sectional view of the shaft of FIG. 3 taken along section 4-4 is shown in accordance with various embodiments herein. In various embodiments, the insulated zone 105 of shaft 104 includes a supply tube 324 concentrically located within a return tube 326, which is concentrically located within an insulating shaft 328. The insulated zone 105 can include a shaft portion of the vacuum chamber 336 and an insulated portion of the working gas circuit 210. In various embodiments, the vacuum chamber 336 surrounds and is isolated from the insulated portion of the working gas circuit 210.

In various embodiments, after exiting the handle 102, the high-pressure flow of the working fluid travels distally down the insulated zone of the shaft through supply tube 324. After cooling and expansion in the expansion chamber 106, the working fluid travels proximally back through the insulated zone 105 of the shaft 104 in the annular space between the supply tube 324 and return tube 326.

In various embodiments, the materials and dimensions of each of the layers of the shaft 104 may be selected to provide sufficient degree of flexibility for the shaft to be bendable about its longitudinal axis at the working temperatures of the device.

In various embodiments, the supply tube 324, which also may be referred to as a capillary tube herein, is constructed from any suitable material or materials such as flexible metals, polymers, composites, or the like. In an embodiment, the supply tube 324 is constructed from Nitinol (NiTi), stainless steel, or the like.

In some embodiments, the inner diameter of the supply tube 324 can be greater than or equal to 0.30 mm, 0.35 mm, 0.40 mm, or 0.45 mm. In some embodiments, the inner diameter of the supply tube 324 can be less than or equal to 0.60 mm, 0.55 mm, 0.50 mm, or 0.45 mm. In some embodiments, the diameter of the supply tube 324 can fall within a range of 0.30 mm to 0.60 mm, or 0.35 mm to 0.55 mm, or 0.40 mm to 0.50 mm, or can be about 0.45 mm.

In some embodiments, the outer diameter of the supply tube 324 can be greater than or equal to 0.38 mm, 0.43 mm, 0.48 mm, 0.53 mm, or 0.58 mm. In some embodiments, the outer diameter can be less than or equal to 0.78 mm, 0.73 mm, 0.68 mm, 0.63 mm, or 0.58 mm. In some embodiments, the outer diameter can fall within a range of 0.38 mm to 0.78 mm, or 0.43 mm to 0.73 mm, or 0.48 mm to 0.68 mm, or 0.53 mm to 0.63 mm, or can be about 0.58 mm.

In some embodiments, the thickness of the supply tube 324 can be greater than or equal to 0.10 mm, 0.11 mm, 0.12 mm, 0.14 mm, or 0.15 mm. In some embodiments, the thickness of the supply tube 324 can be less than or equal to 0.20 mm, 0.19 mm, 0.18 mm, 0.16 mm, or 0.15 mm. In some embodiments, the thickness of the supply tube 324 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the return tube 326 is constructed from any suitable material or materials such as flexible metals, polymers, or the like. In various embodiments, the return tube 326 can be made of polyimide, fluorinated ethylene propylene (FEP), Teflon, or the like. In an embodiment, the return tube 326 is formed from a polyimide material as it is highly impermeable to gases at a wide range of temperatures and can thus contain the working fluid inside and hold vacuum on the outside. In a particular example, the return tube 326 is made of a braid-reinforced polyimide tube to enhance gas impermeability, burst strength, and flexibility. In some embodiments, the return tube 326 is formed from a single layer of material. In some embodiments, the return tube 326 can be formed from two or more layers of material selected to optimize the performance of the shaft 104. The layers of material can be bonded together using any suitable technique or techniques such as adhesives, reflow processes, or the like.

In some embodiments, the outer diameter of the return tube 326 can be greater than or equal to 1 mm, 1.1 mm, 1.2 mm, 1.3 mm, or 1.4 mm. In some embodiments, the outer diameter of the return tube 326 can be less than or equal to 1.8 mm, 1.7 mm, 1.6 mm, 1.5 mm, or 1.4 mm. In some embodiments, the outer diameter of the return tube 326 can fall within a range of 1.0 mm to 1.8 mm, or 1.1 mm to 1.7 mm, or 1.2 mm to 1.6 mm, or 1.3 mm to 1.5 mm, or can be about 1.4 mm.

In some embodiments, the inner diameter of the return tube 326 can be greater than or equal to 0.9 mm, 1.0 mm, 1.1 mm, 1.2 mm, or 1.3 mm. In some embodiments, the inner diameter of the return tube 326 can be less than or equal to 1.7 mm, 1.6 mm, 1.5 mm, 1.4 mm, or 1.3 mm. In some embodiments, the inner diameter of the return tube 326 can fall within a range of 0.9 mm to 1.7 mm, or 1.0 mm to 1.6 mm, or 1.1 mm to 1.5 mm, or 1.2 mm to 1.4 mm, or can be about 1.3 mm.

In some embodiments, the thickness of the return tube 326 can be greater than or equal to 0.10 mm, 0.11 mm, 0.12 mm, 0.14 mm, or 0.15 mm. In some embodiments, the thickness of the return tube 326 can be less than or equal to 0.20 mm, 0.19 mm, 0.18 mm, 0.16 mm, or 0.15 mm. In some embodiments, the thickness of the return tube 326 can fall within a range of 0.10 mm to 0.20 mm, or 0.11 mm to 0.19 mm, or 0.12 mm to 0.18 mm, or 0.14 mm to 0.16 mm, or can be about 0.15 mm.

In various embodiments, the insulating shaft 328 is constructed from any suitable material or materials such as flexible metals, polymers, or the like. In various embodiments, the insulating shaft 328 is made of polyimide, fluorinated ethylene propylene (FEP), Teflon, or the like. In a particular embodiment, the insulating shaft 328 may include polytetrafluoroethylene (PTFE), and/or one or more polyether block amides (known under the tradename Pebax®, hereinafter “Pebax”).

In some embodiments, the insulating shaft 328 is formed from a single layer of material. In some embodiments, the insulating shaft 328 can be formed from two or more layers of material selected to optimize the performance of the shaft 104. The layers of material can be bonded together using any suitable technique or techniques such as adhesives, reflow processes, or the like.

In an embodiment, the insulating shaft may be formed using a braid-reinforced polyimide tube skim-coated with a Pebax outer layer. Such a tri-layer construction enables the deep vacuum to be maintained between the return tube and the insulating shaft without causing the insulating shaft 328 to collapse onto the return tube 326.

In some embodiments, the outer diameter of the insulating shaft 328 can be greater than or equal to 1.2 mm, 1.4 mm, 1.5 mm, 1.6 mm, or 1.8 mm. In some embodiments, the outer diameter of the insulating shaft can be less than or equal to 2.2 mm, 2.1 mm, 2.0 mm, 1.9 mm, or 1.8 mm. In some embodiments, the outer diameter of the insulating shaft can fall within a range of 1.3 mm to 2.3 mm, or 1.4 mm to 2.1 mm, or 1.5 mm to 2.0 mm, or 1.6 mm to 1.9 mm, or can be about 1.8 mm.

In some embodiments, the inner diameter of the insulating shaft 328 can be greater than or equal to 1.0 mm, 1.2 mm, 1.3 mm, 1.4 mm, or 1.6 mm. In some embodiments, the inner diameter of the insulating shaft 328 can be less than or equal to 2.2 mm, 2.0 mm, 1.9 mm, 1.8 mm, or 1.6 mm. In some embodiments, the inner diameter of the insulating shaft 328 can fall within a range of 1.0 mm to 2.2 mm, or 1.2 mm to 2.0 mm, or 1.3 mm to 1.9 mm, or 1.4 mm to 1.8 mm, or can be about 1.6 mm.

In some embodiments, the thickness of the insulating shaft 328 can be greater than or equal to 0.10 mm, 0.11 mm, 0.12 mm, 0.14 mm, or 0.15 mm. In some embodiments, the thickness of the insulating shaft 328 can be less than or equal to 0.20 mm, 0.19 mm, 0.18 mm, 0.16 mm, or 0.15 mm. In some embodiments, the thickness of the insulating shaft 328 can fall within a range of 0.10 mm to 0.20 mm, or 0.11 mm to 0.19 mm, or 0.12 mm to 0.18 mm, or 0.14 mm to 0.16 mm, or can be about 0.15 mm.

In some embodiments, a PEEK filament 330 is wound around the return tube 326. PEEK filament 330 may have pitch that is greater than or equal to 0.5 mm, 1.0 mm, 1.5 mm, or 2.0 mm, or can be an amount falling within a range between any of the foregoing. Alternatively, the filament may be a plurality of discrete pieces attached along the return tube 326. The PEEK filament 330 prevents direct contact between the return tube 326 and the insulating shaft 328, maintaining their coaxial alignment. In some embodiments, an adhesive (e.g., Loctite) is applied on the filament at the end of the return tube 326 and the insulating shaft 328 to attach the PEEK filament 330. In various embodiments, the PEEK filament wrap is configured to minimize or prevent conductive heat transfer from the return tube to the insulating shaft. In alternative embodiments, other insulating polymers may be used as a substitute for the PEEK filament such as expanded PTFE (ePTFE), nylon, or the like.

In some embodiments, the diameter of the PEEK filament 330 can be greater than or equal to 0.002 mm, 0.004 mm, or 0.005 mm. In some embodiments, the diameter of the PEEK filament 330 can be less than or equal to 0.007 mm, 0.006 mm, or 0.005 mm. In some embodiments, the diameter of the PEEK filament 330 can fall within a range of 0.002 mm to 0.007 mm, or 0.004 mm to 0.006 mm, or can be about 0.005 mm.

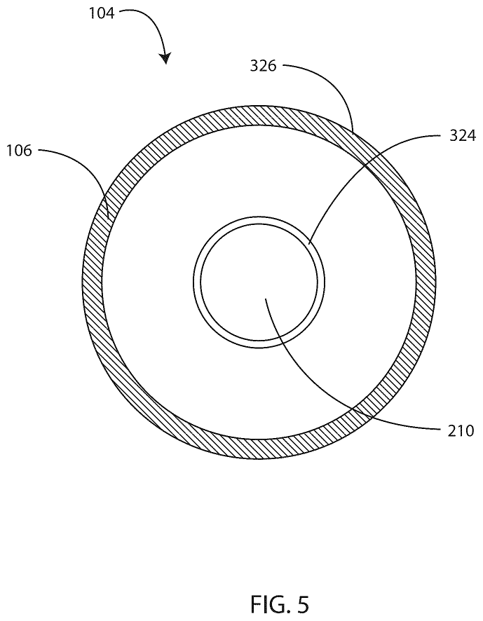

### Shaft in the Expansion Chamber (FIG. 5)

Referring now to FIG. 5, a cross-sectional view of the shaft of FIG. 3 taken along section 5-5 is shown in accordance with various embodiments herein. The cross-sectional view of FIG. 5 depicts expansion chamber 106 of the shaft 104. In various embodiments, the expansion chamber 106 is distal to the insulated zone 105 along the shaft 104. The expansion chamber 106 can include an expansion portion of the working fluid circuit 210.

In various embodiments, after exiting the handle 102, the high-pressure flow of the working fluid travels down the supply tube 324. After cooling and expansion in expansion chamber 106, the working fluid travels back down through the expansion chamber 106 in the annular space between the supply tube 324 and the outer wall of the expansion chamber. In various embodiments, the expansion chamber 106 is configured to maximize the heat transfer between the working gas and the patient's tissue through the optimization of parameters such as wall thickness, materials, and the like.

In various embodiments, the expansion chamber 106 is constructed from any suitable material or materials such as flexible metals, polymers, or the like. In various embodiments, the expansion chamber 106 is made of polyimide, fluorinated ethylene propylene (FEP), Teflon, or the like. In some embodiments, the expansion chamber 106 includes a continuation of the return tube 326 of the insulated zone 105 of the shaft 104. Alternatively, the expansion chamber is a separate component from the return tube 326 that can be joined to the shaft 104 using any suitable joint and/or fitting, such as reflow processes, glue joints, solder joints, or any other suitable mechanical joining process capable of withstanding cryogenic pressures and temperatures.

In some embodiments, the expansion chamber 106 is formed from a single layer of material. In some embodiments, the expansion chamber 106 is formed from two or more layers of material. The layers of material can be bonded together using any suitable technique or techniques such as adhesives, reflow processes, or the like.

In some embodiments, the outer diameter of the expansion chamber 106 can be greater than or equal to 1.3 mm, 1.4 mm, 1.5 mm, 1.6 mm, or 1.7 mm. In some embodiments, the outer diameter of the expansion chamber 106 can be less than or equal to 2.1 mm, 2.0 mm, 1.9 mm, 1.8 mm, or 1.7 mm. In some embodiments, the outer diameter of the expansion chamber 106 can fall within a range of 1.3 mm to 2.1 mm, or 1.4 mm to 2.0 mm, or 1.5 mm to 1.9 mm, or 1.6 mm to 1.8 mm, or can be about 1.7 mm.

In some embodiments, the inner diameter of the expansion chamber 106 can be greater than or equal to 1.0 mm, 1.1 mm, 1.2 mm, 1.3 mm, or 1.4 mm. In some embodiments, the inner diameter of the expansion chamber 106 can be less than or equal to 1.8 mm, 1.7 mm, 1.6 mm, 1.5 mm, or 1.4 mm. In some embodiments, the inner diameter of the expansion chamber 106 can fall within a range of 1.0 mm to 1.8 mm, or 1.1 mm to 1.7 mm, or 1.2 mm to 1.6 mm, or 1.3 mm to 1.5 mm, or can be about 1.4 mm. In some embodiments, the thickness of the wall of expansion chamber 106 can be greater than or equal to 0.20 mm, 0.22 mm, 0.25 mm, 0.28 mm, or 0.30 mm. In some embodiments, the thickness of the wall of the expansion chamber 106 can be less than or equal to 0.40 mm, 0.38 mm, 0.35 mm, 0.32 mm, or 0.30 mm. In some embodiments, the thickness of the wall of the expansion chamber 106 can fall within a range of 0.20 mm to 0.40 mm, or 0.22 mm to 0.38 mm, or 0.25 mm to 0.35 mm, or 0.28 mm to 0.32 mm, or can be about 0.30 mm.

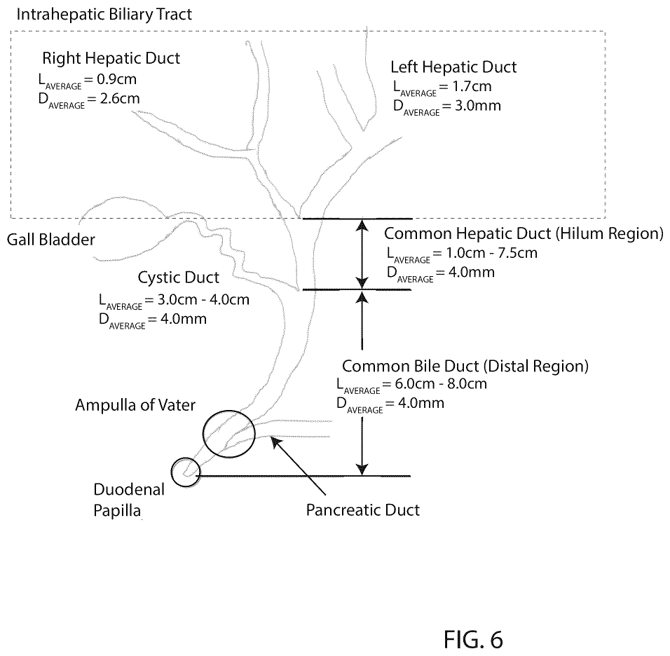

### Treatment Area Examples (FIG. 6)

The cryoablation systems and structures described herein are flexible and well-suited to cryoablate an area along a lumen, vessel, or passageway of the body. The present cryoablation system 100 is configured to be adequately flexible to access and ablate many different such structures. The expansion chamber may be shaped and sized to generate an ice ball or ice cylinder of appropriate geometry for ablating structures along the length of a body lumen. Moreover, the materials and configuration of the probes described herein are selected to protect patient tissue and withstand high operating pressures and low temperatures.

Referring now to FIG. 6, a schematic view of the biliary system is shown in accordance with various embodiments herein. The biliary system includes organs and ducts that make and store bile (a fluid made by the liver that helps digest fat) and release it into the small intestine. The biliary system includes the gallbladder and bile ducts inside and outside the liver.

Bile duct cancer or cholangiocarcinoma is a rare disease in which cancer cells form in the bile ducts. Treatment outcomes for cholangiocarcinoma are generally poor. Current treatment options such as a Whipple procedure or biliary drains are high risk and often ineffective.

Cryoablation is a promising treatment for cholangiocarcinoma. The present cryoablation system 100 is configured to be adequately flexible to access and ablate the bile ducts of a patient. The expansion chamber may be shaped and sized to generate an ice ball of appropriate geometry for ablating tumors in the bile ducts.

In addition to the treatment of cholangiocarcinoma, the cryoablation system 100 can be used to treat a number of conditions including other cancerous tumors (e.g., skin, liver, kidney, bone, lung, prostate and breast), pain, skin conditions (e.g., atypical moles, warts, skin tags or actinic keratosis), arrythmia, or the like. The cryoablation system 100 can additionally ablate benign masses, soft tissue, and healthy tissue.

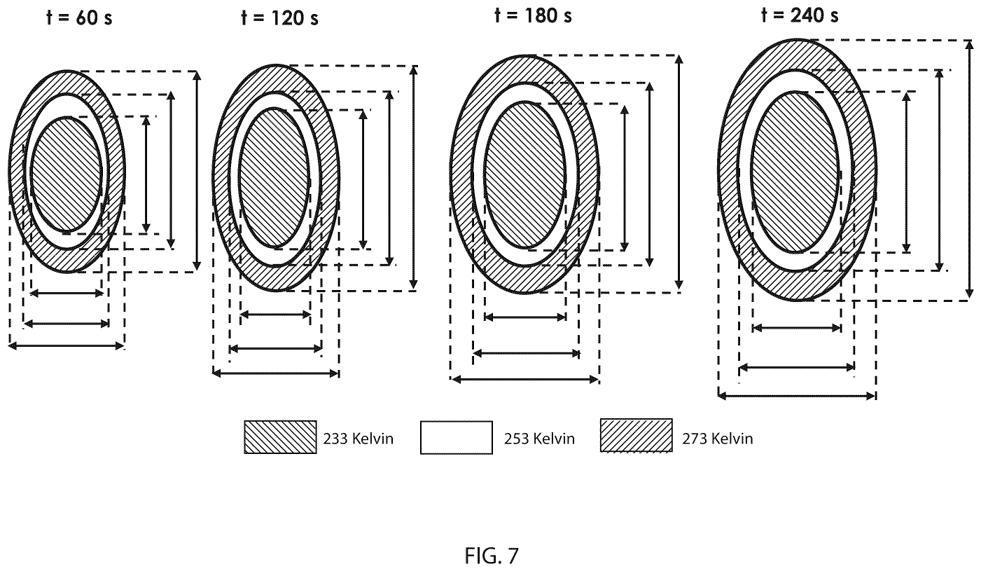

### Ice Ball Formation (FIG. 7)

Referring now to FIG. 7, a schematic view of the growth of an ice ball generated by a cryoablation system is shown in accordance with various embodiments herein. Cryoablation is defined as cell destruction using cold temperatures. An ice ball is formed at the expansion chamber of a cryoablation system, which freezes intra cellular and extracellular material to temperatures colder than 173 Kelvin. The application of these extremely cold temperatures causes cell death. In order to cause complete cell death, sufficiently low ablation temperatures are be achieved. Lethal temperatures for various tissues have been reported between 253 Kelvin and 233 Kelvin.

FIG. 7 illustrates the growth of an ice ball over the course of a cryoablation procedure. The time points in this example are at 60 seconds, 120 seconds, 180 seconds, and 240 seconds. Isotherms for 273 Kelvin, 253 Kelvin and 233 Kelvin are depicted for each period of time. As the cryoablation procedure progresses in time, the overall ice ball size increases. More critically, the cell killing isotherms (the 253 Kelvin and 233 Kelvin isotherms) grow along both the major and minor axes of the ice ball.

It should be noted that the times and temperatures given by FIG. 7 are for exemplary purposes only. Ice ball formation can vary based on the configuration of the cryoablation system and the patient's tissue. Moreover, ice ball growth can stabilize after a certain ablation time has been reached. For example, in some embodiments, an ice ball can reach its maximum diameter after ablating the tissue for approximately 10 minutes. In some embodiments, the cryoablation system operator can terminate a cryoablation procedure upon the ice ball reaching its maximum size. In some embodiments, the cryoablation system operator can continue the cryoablation procedure upon the ice ball reaching its maximum size for a predetermined amount of time, as holding the tissue at cryogenic temperatures for longer times may have therapeutic benefits.

In some embodiments, the ice ball is produced and then thawed, and then produced again. In some embodiments, active thawing techniques are used while in other embodiments passive thawing is used.

In the example of FIG. 7, the ice balls are oblong in shape (e.g., they are longer along a primary axis than a secondary axis). The primary axis of the ice ball corresponds to a lengthwise axis of the expansion chamber. The ratio of expansion chamber height to diameter can be correlated to ice ball shape. In particular, an expansion chamber that is much longer than it is wide will result in a more oblong ice ball. Such an ice ball is generally more suitable for the treatment of cholangiocarcinoma, as it is more compatible with the anatomy of the bile ducts. However, other cryoablation applications may require a more spherical ice ball. In such scenarios, the expansion camber can be modified to have a smaller length to diameter ratio.

In some embodiments, the major axis length of an ice ball can be greater than or equal to 0.5 mm, 2 mm, 4 mm, 6 mm, 8 mm, or 10 mm, or can be an amount falling within a range between any of the foregoing. In some embodiments, the minor axis length of an ice ball can be greater than or equal to 0.5 mm, 2 mm, 4 mm, 6 mm, 8 mm, or 10 mm, or can be an amount falling within a range between any of the foregoing.

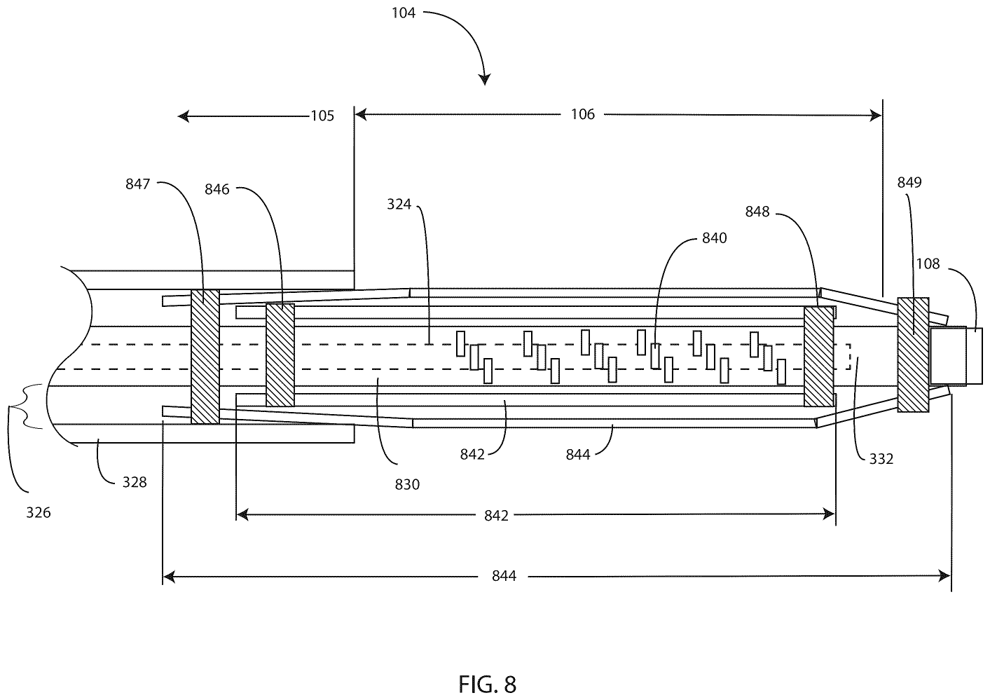

### Cryoablation Shaft Including a Slotted Tube Portion, a Polymer Layer, and a Reinforcing Layer (FIG. 8)

Referring now to FIG. 8, a schematic view of a tip portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 concentrically located within a return tube 326, which is concentrically located within an insulating shaft 328. The concentric-shaft construction is designed to isolate the working gas and active vacuum circuits from each other.

In various embodiments, after exiting the handle 102, the high-pressure flow of the working fluid travels down the supply tube 324. The high-pressure stream of the working fluid may undergo expansion at or downstream of the Joule-Thomson orifice 332 and returns down the shaft in the annular space between the supply tube 324 and the return tube 326.

In various embodiments, the expansion chamber 106 of shaft 104 includes a supply tube 324 concentrically located within the return tube 326. The return tube 326 includes multiple layers in various embodiments.

In various embodiments, the innermost layer of the return tube is a slotted tube 830 which includes slots 840 along at least a portion of the length of shaft 104. Slots 840 are formed in the tube material by any suitable means, such as laser cutting. The slots 840 can be laser cut into the shaft using any suitable pattern to optimize the strength and flexibility of the return tube 326. The material of the slotted tube 830 can be metal such as stainless steel, nitinol, or other durable materials. Many different configurations and patterns of slotted tube 830 are available and one can be selected with the flexibility desirable in this application. The slotted tube forms the core of the return tube 326 in various embodiments.

In one embodiment, the slotted tube 830 includes the slots only in the expansion chamber 106. In this embodiment, the portion of the slotted tube 830 that extends through the insulated zone 105 has solid walls. FIG. 8 illustrates this embodiment by showing a schematic side view of the slotted tube 830, including slots 840 in the expansion chamber 106, and showing the supply tube 324 in dashed lines within the slotted tube 830. In an alternative embodiment, the slotted tube 830 has slots along its entire length.

The slotted tube may be configured to be sufficiently flexible to form a curve having a desired radius of curvature. For instance, the slotted tube may be sufficiently flexible, such that the shaft may form a curve having the smallest radius of curvature of less than or equal to 30 mm, 20 mm, 10 mm, 5 mm, or 3 mm. The slotted tube 830 may have different levels of flexibility in the expansion chamber and the insulated zone 105.

In various embodiments, a first polymer layer 842 surrounds the portion of the slotted tube 830 which includes the slots, in order to contain the working fluid within the shaft 104. In various embodiments, a reinforcing layer 844 surrounds the first polymer layer 842 and is configured to provide additional strength and reinforcement to the first polymer layer 842, thus reducing the likelihood of any leak or rupture in the first polymer layer.

In various embodiments, the first polymer layer 842 can be formed from any suitable polymer such as polyethylene terephthalate (PET), PTFE, ePTFE, PEEK, polyetherimide (PEI), polyimide (PI), or the like. In various embodiments, the reinforcing layer 844 can be a second polymer layer formed from any suitable polymer such as PET, PTFE, PEEK, polyetherimide (PEI), polyimide (PI), or the like. In various embodiments, the reinforcing layer is gas impermeable. In various embodiments, the reinforcing layer 844 is not impermeable and can include a braided polymer material and/or a coiled polymer or metallic material and/or coatings & encapsulants.

In various embodiments, the return tube may include two, three, four, or more polymer layers. In various embodiments, the return tube may include two, three, four, or more total layers.

In the embodiment of FIG. 8, where the slotted portion of the slotted tube 830 is within the expansion chamber, the first polymer layer 842 and the reinforcing layer 844 extend from just within the insulated zone 105 to the distal end. In various embodiments, the first polymer layer 842 is bonded to the slotted tube 830 at first bond 846 near the proximal end of the expansion chamber 106 and at a second bond 848 near the tip 108 of the shaft 104.

In various embodiments, the reinforcing layer 844 is bonded to the slotted tube 830 at first bond 847 near the proximal end of the expansion chamber 106 and/or at a second bond 849 near the tip 108 of the shaft 104.

Additional bonds can be placed along any other suitable location of the shaft. The bonds can be formed from any suitable material or materials such as Vectran, UHMWPE, PEEK, Polyimide, Metallic Wire. Vectran is a manufactured fiber, spun from a liquid-crystal polymer that displays increased tensile strength at cold temperatures, making it advantageous for use in cryoablation systems. In alternative embodiments, the bonds 846, 847, 848 and 849 may be formed from alternate techniques such as crimp or swage rings, polymer reflow joints, adhesive joints, solder joints or the like.

In various embodiments, the wrap materials of the bonds 846, 847, 848 and 849, can also be wrapped along the entire length of the expansion chamber around the polymer layer, the reinforcing layer, or both. The wrap layer may have a higher pitch of wraps at the bond location and extend along the remainder of the expansion chamber at a lower pitch level. The wrap may extend from the proximal end to the distal end and then reverse direction and extend back in a proximal direction. The number of wrap layers may be one, two, three, four or more.

In various embodiments, the bonds 846, 847, 848 and 849 are configured to increase the burst strength of the layers of the return tube 326.

In some embodiments, the burst strength of each of the first polymer layer 842 and the reinforcing layer 844 can be greater than or equal to 12.4 MPa, 13.1 MPa, 13.8 MPa, 14.5 MPa, 15.2 MPa, or 41.4 MPa. In some embodiments, the burst strength of each of the first polymer layer 842 and the reinforcing layer can be less than or equal to 41.4 MPa, 20.7 MPa, 19.3 MPa, 17.9 MPa, 16.5 MPa, or 15.2 MPa. In some embodiments, the burst strength can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of the bonded first polymer layer 842 and the reinforcing layer 844 together can be greater than or equal to 12.4 MPa, 13.1 MPa, 13.8 MPa, 14.5 MPa, 15.2 MPa 27.6 MPa, or 41.4 MPa. In some embodiments, the burst strength of the bonded first polymer layer 842 and the reinforcing layer 844 together can be less than or equal to 41.4 MPa, 20.7 MPa, 19.3 MPa, 17.9 MPa, 16.5 MPa, or 15.2 MPa. In some embodiments, the burst strength of the bonded first polymer layer 842 and the reinforcing layer 844 together can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of each of the bonds 846, 847, 848 and 849 can be greater than or equal to 1.4 MPa, 4.8 MPa, 8.3 MPa, 11.7 MPa, 15.2 MPa, 27.6 MPa, or 41.4 MPa. In some embodiments, the burst strength of each of the bonds can be less than or equal to 41.4 MPa, 34.8 MPa, 28.3 MPa, 21.7 MPa, or 15.2 MPa. In some embodiments, the burst strength of each of the bonds can fall within a range of 1.4 MPa to 41.4 MPa, or 4.8 MPa to 34.8 MPa, or 8.3 MPa to 28.3 MPa, or 11.7 MPa to 21.7 MPa, or can be about 15.2 MPa.

In some embodiments, the bonds 846, 847, 848 and 849 can have the same burst strength. Alternatively, some of the bonds can have a higher burst strength than others. For instance, proximal bonds 846, 847 may have a lower burst strength than distal bonds 848, 849.

In various embodiments, the return tube 326 may be configured to be sufficiently flexible to form a curve having a desired radius of curvature. For instance, the return tube may be sufficiently flexible, such that the shaft may form a curve having the smallest radius of curvature of less than or equal to 30 mm, 20 mm, 10 mm, 5 mm, or 3 mm.

### Slotted Tube Portion can Extend the Entire Length of the Return Tube

FIG. 8 illustrates that the slotted tube 830 includes the slots only in the expansion chamber 106, but in an alternative embodiment, the slotted tube 830 has slots along its entire length. In this alternative embodiment, the first polymer layer 842 and the reinforcing layer 844 will also extend along the entire length of the return tube 326. The reinforcing wrap layer, such as a wrap of a Vectran material, may also extend along the entire return tube length. The presence of slots along the entire return tube length may be desirable to achieve the desired level of flexibility along the return tube.

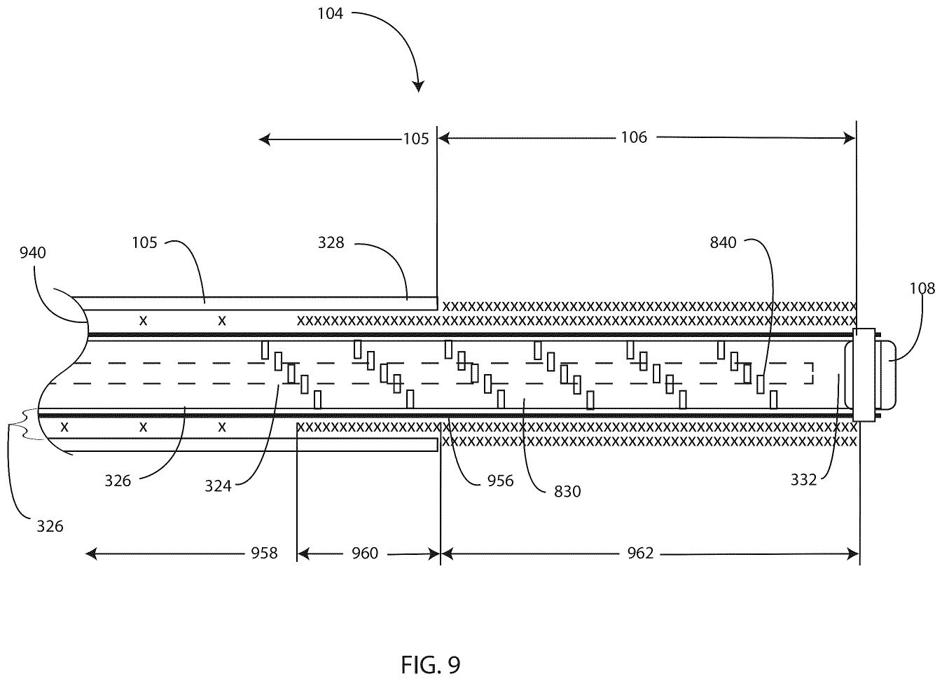

### Cryoablation Shaft Including a Slotted Tube Portion and a Gradient Braid (FIG. 9)

Referring now to FIG. 9, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 located within a return tube 326. The return tube 326 of FIG. 9 includes multiple layers.

The innermost layer of the return tube of 326 is a slotted tube 830, which can be constructed using the options and details described herein. The slotted tube 830 is surrounded by a polymer layer 956, which is concentrically surrounded by a reinforcing gradient braid layer 940.

In various embodiments, gradient braid layer 940 includes zones of different density of braid, increasing in density toward the distal end of the device. A first braid zone 958 is present in the insulated zone and is the least dense. A second braid zone 960 overlaps the insulated zone 105 and the expansion chamber 106 and is more dense than the first braid zone 958. The third braid zone 962 is the densest and is present in the expansion zone. The return tube may include one, two, three, or more braid layers.

In various embodiments, the polymer layer 956 is configured to contain the working fluid in the return tube and not allow the working fluid to escape radially through the slots in the slotted tube. The polymer layer may be constructed from the options and materials discussed herein with respect to the first polymer layer herein. In some embodiments, the expansion chamber 106 may include an additional braided layer between the slotted tube 830 and the polymer layer 956 (not shown in this view). The additional braided layer is configured to prevent friction between the polymer layer 956 and the slotted tube 830.

### Braid Materials, Braid Element Diameters, Braid Density Zones, Coils, and Burst Strength Examples

Braided tubes are used in a variety of medical applications. Braid reinforced tubing can improve functional properties such as strength, stiffness, burst pressure resistance, torque transmission, and kink resistance of a medical device. Such features enable can enable a cryoablation shaft to navigate tortuous portions of a patient's anatomy, such as the biliary ducts. Design considerations such as braid pattern, pick count (ppi), material, wire dimension, wire size/shape and durometer of plastics can have significant impact on device performance.

The braided portions of the shaft can be formed from any suitable material or materials such as metals (e.g., nitinol, stainless steel, tungsten, MP35N, or other such materials), polymers, (e.g., PET, Kevlar, Carbon fiber, Vectran, or other such materials), or the like. Braided materials are formed by weaving a metal or fiber filament in a braid pattern. In various embodiments, the cross section of the filament is circular, however other cross-sectional shapes are possible (e.g., flat, star, triangle). In some embodiments, the diameter of the filament can be greater than or equal to 0.01 mm, 0.1 mm, 0.2 mm, 0.3 mm, or 0.5 mm. In some embodiments, the diameter of the filament can be less than or equal to 2 mm, 1.6 mm, 1.2 mm, 0.8 mm, or 0.5 mm. In some embodiments, the diameter of the filament can fall within a range of 0.01 mm to 2.00 mm, or 0.1 mm to 1.6 mm, or 0.2 mm to 1.2 mm, or 0.3 mm to 0.8 mm, or can be about 0.5 mm.

In various embodiments, the density of the braided portions can be altered with more dense braids offering more radial strength and rigidity and less dense braids offering more flexibility. The shaft can include multiple braids having varying material properties. In various embodiments, the shaft may include a light braid (e.g., have relatively low braid density) between the return tube 326 and the polymer layer 956. As described above, the light braid may offer limited structural support but can prevent undue friction between the slotted return tube 326 and the polymer layer 956.

In various embodiments, the density of the braided materials can increase from the base of the shaft 104 to the tip 108 of the shaft. In the event that there is a device failure, such a configuration improves the likelihood of the shaft failing closer to the base. Such a failure mode is generally preferable compared to a failure closer to the tip 108 of the shaft in terms of patient safety outcomes.

In various embodiments, the first gradient braid zone 958 spans the insulated zone 105 of the shaft 104 (starting from where the shaft connects to the handle 102 and terminating at or before the expansion chamber 106). In various embodiments, the gradient braid 958 portion can have a first burst strength. The first burst strength can be constant along the insulated zone of the shaft. Alternatively, the first burst strength of the gradient braid portion 958 can increase from the base of the shaft to the expansion chamber. In various embodiments, the gradient braid portion 958 can have a first braid density. The first braid density can be constant along the insulated zone of the shaft. Alternatively, the first braid density of the gradient braid portion 958 can increase from the base of the shaft to the expansion chamber.

In some embodiments, the minimum burst strength of the gradient braid portion 958 can be less than or equal to 20.7 MPa, 13.8 MPa, 6.9 MPa, 5.5 MPa, 4.1 MPa, 2.8 MPa, 1.4 MPa, or 0.7 MPa, or can be an amount falling within a range between any of the foregoing. In some embodiments, the maximum burst strength of the gradient braid portion 958 can be greater than or equal to 0.7 MPa, 2.0 MPa, 3.4 MPa, 4.8 MPa, 6.2 MPa, 6.9 MPa, 13.8 MPa, or 20.7 MPa or can be an amount falling within a range between any of the foregoing.

In various embodiments, the second braid portion 960 spans the expansion chamber 106 of the shaft 104. In some embodiments, the second braid portion 960 may start at the beginning of the expansion chamber and terminate at or near the tip 108 of the shaft. Alternatively, the second braid portion 960 may start towards the distal end of the insulated zone 105 of the shaft (as depicted by FIG. 9) and terminate at or near the tip 108 of the shaft. In some embodiments, the second braid portion 960 is a continuation of the first braid portion 958. Alternatively, the second braid portion 960 may be made of elements that are physically separate from the elements of first braid zone 958.

In various embodiments, the second braid portion 960 can have a second burst strength. The second burst strength can be constant along the length of the first braid layer. Alternatively, the second burst strength of the second braid portion 960 can increase from the along the length of the first braid layer (from the end of the insulated zone 105 to the tip 108). In various embodiments, the second braid portion 960 can have a second braid density. The second braid density can be constant along the length of the second braid portion. Alternatively, the second braid density of the second braid portion 960 can increase along the length of the second braid portion.

In some embodiments, the burst strength of the second braid portion 960 can be greater than or equal to 10.3 MPa, 12.9 MPa, 15.5 MPa, 18.1 MPa, or 20.7 MPa, or 41.4 MPa. In some embodiments, the burst strength of the second braid portion 960 can be less than or equal to 34.5 MPa, 31.0 MPa, 27.6 MPa, 24.1 MPa, or 20.7 MPa. In some embodiments, the burst strength of the second braid portion 960 can fall within a range of 10.3 MPa to 34.5 MPa, or 12.4 MPa to 31.0 MPa, or 15.5 MPa to 27.6 MPa, or 17.9 MPa to 34.5 MPa, or can be about 20.7 MPa.

In various embodiments, the third braid zone 962 spans the expansion chamber 106 of the shaft 104. In some embodiments, the third braid zone 962 may start at the beginning of the expansion chamber and terminate at or near the tip 108 of the shaft.

In various embodiments, the third braid zone 962 can have a third burst strength. The third burst strength can be constant along the length of the third braid zone. Alternatively, the burst strength of the third braid zone 962 can increase from the along the length of the third braid zone. In various embodiments, the third braid zone 962 can have a third braid density. The braid density can be constant along the length of the third braid zone. Alternatively, the braid density of the third braid zone 962 can increase along the length of the first braid layer.

In some embodiments, the burst strength of the third braid zone 962 can be greater than or equal to 10.3 MPa, 12.9 MPa, 15.5 MPa, 18.1 MPa, or 20.7 MPa. In some embodiments, the burst strength of the third braid zone 962 can be less than or equal to 34.5 MPa, 31.0 MPa, 27.6 MPa, 24.1 MPa, or 20.7 MPa. In some embodiments, the burst strength of the third braid zone 962 can fall within a range of 10.3 MPa to 34.5 MPa, or 12.4 MPa to 31.0 MPa, or 15.5 MPa to 27.6 MPa, or 17.9 MPa to 24.1 MPa, or can be about 20.7 MPa.

In alternative embodiments, any, or all of the gradient braid zones 958, 960, 962 can be formed from coils. A coil, as defined herein is a filament of material wound around the shaft. Like in braided materials, as detailed above, the filament can be selected to have suitable material(s) and cross-sectional shapes including round, rectangular, or other shapes. The zones can include coils of different density, radial strength, rigidity, and flexibility. The coils can be single layered or multi layered. In some embodiments, the multi-layer coils can have alternate wind directions (e.g., first layer is wound clockwise, and the second layer is wound counterclockwise). In various embodiments, the pitch of the coil can be varied to optimize the properties of the shaft, such as flexibility, burst strength, or the like. For instance, tighter pitches of coil can increase the burst strength of the shaft while looser pitches of coil can increase the flexibility of the shaft. In various embodiments, the pitch of the coil materials can increase from the base of the shaft 104 to the tip 108 of the shaft.

### Transitions Between Braid Zones

In some embodiments, the transitions between the first, second and third braid portions are transitions in density of the same physical braid elements, so that one braid zone is a continuation of a neighboring braid zone. Alternatively, one braid portion may be made of elements that are physically separate from the elements of a neighboring braid zone.

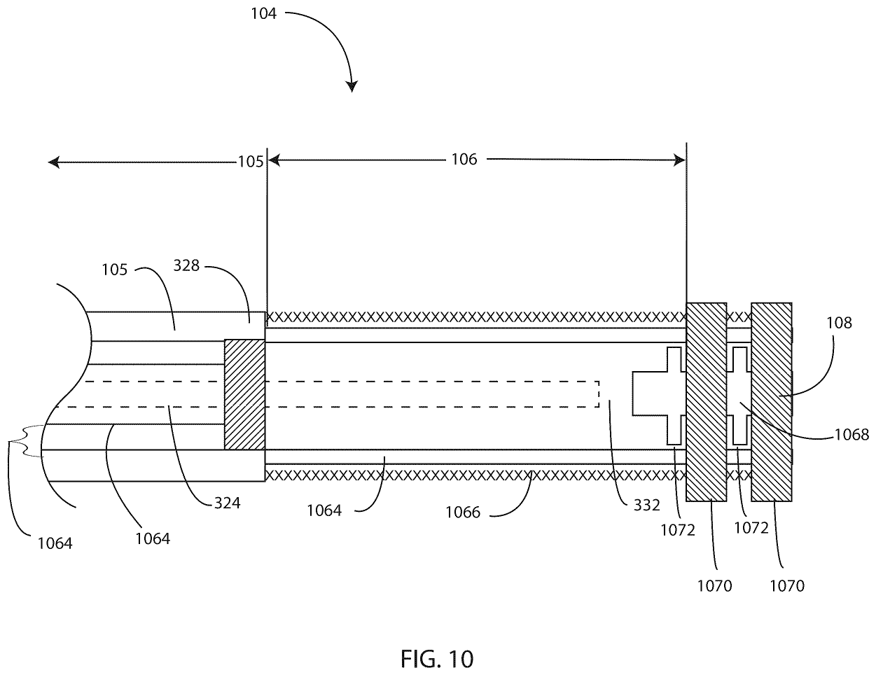

### Composite Shaft (FIG. 10)

Referring now to FIG. 10, a schematic view of a portion of a cryoablation shaft is shown in accordance with various embodiments herein. In various embodiments, the expansion chamber 106 of shaft 104 includes a supply tube 324, located concentrically within a composite return tube 1064 In various embodiments, the return tube can be a composite shaft. In various embodiments, the composite return tube 1064 is formed from one or more discrete layers. One possible return tube layer is a braid layer 1066 shown in FIG. 10.

In various embodiments, composite return tube 1064 can be formed from one or more polymer and/or braided layers as described in detail herein. After forming the discrete layers, the composite shaft may be heated to such a temperature that the discrete layers bond to each other forming a single composite layer. In some embodiments, some of the discrete layers may contribute to the radial strength of the expansion chamber and other discrete layers may contribute to the gas containment capability of the expansion chamber. In such embodiments, radial strength and gas containment may be achieved from a single composite layer. Such a configuration can enhance the tunability of the expansion chamber material properties while decreasing the diameter of the expansion chamber.

In some embodiments, an additional braid layer 1066 may be disposed over the remainder of the composite return tube 1064. The additional braid layer is selected to have braid properties that enhance the radial strength of the expansion chamber 106.

### Distal Fitting Examples (FIG. 10)

In various embodiments, the working fluid may be contained at the tip 108 of the shaft using a plug 1068 and one or more joints 1070. In various embodiments, the joints 1070 and/or the plug 1068 may be of a material or materials that are compatible to bond with the composite return tube 1064 and/or the braid layer 1066 to enhance the seal of the shaft 104. In various embodiments, the joints 1070 and/or the plug 1068 may be of a material or materials that are sufficiently strong as to enhance the burst strength of the expansion chamber.

In various embodiments, plug 1068 includes ridges 1072. The ridges 1072 improve the mechanical strength of the bonded joints 1070 by providing additional structural support to the bonds to resist the pressure within the expansion chamber.

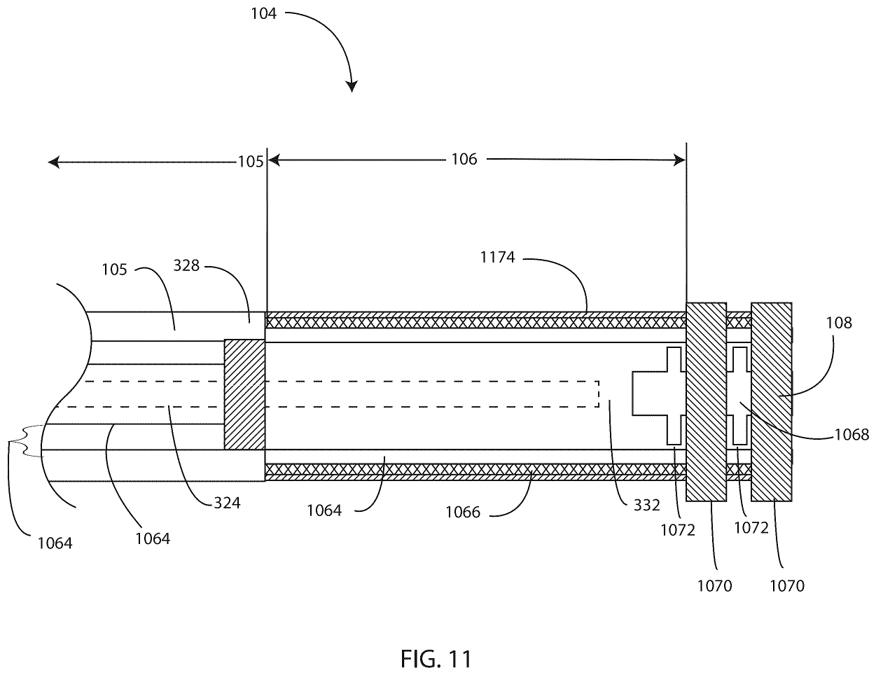

### Composite Shaft—Alternate Configuration (FIG. 11)

Referring now to FIG. 11, a schematic view of a portion of a cryoablation shaft 104 is shown in accordance with various embodiments herein. In various embodiments, the expansion chamber 106 of shaft 104 includes a supply tube 324, located concentrically within a composite return tube 1064.

The composite return tube 1064 of FIG. 11 can have similar layers and/or properties to the composite return tube of FIG. 10. In the example of FIG. 11, an outer polymer layer 1174 may be disposed over the outermost layer of the composite return tube 1064. The outer polymer layer 1174 can be formed from any suitable polymer or blend of polymers such as Pebax, polyethylene terephthalate (PET), PTFE, ePTFE, PEEK, polyetherimide (PEI), polyimide (PI), or the like. The outer polymer layer 1174 can improve the functionality of the shaft 104 by providing any of increased burst strength, biocompatibility, durability over cryoablation temperatures and pressures, or the like.

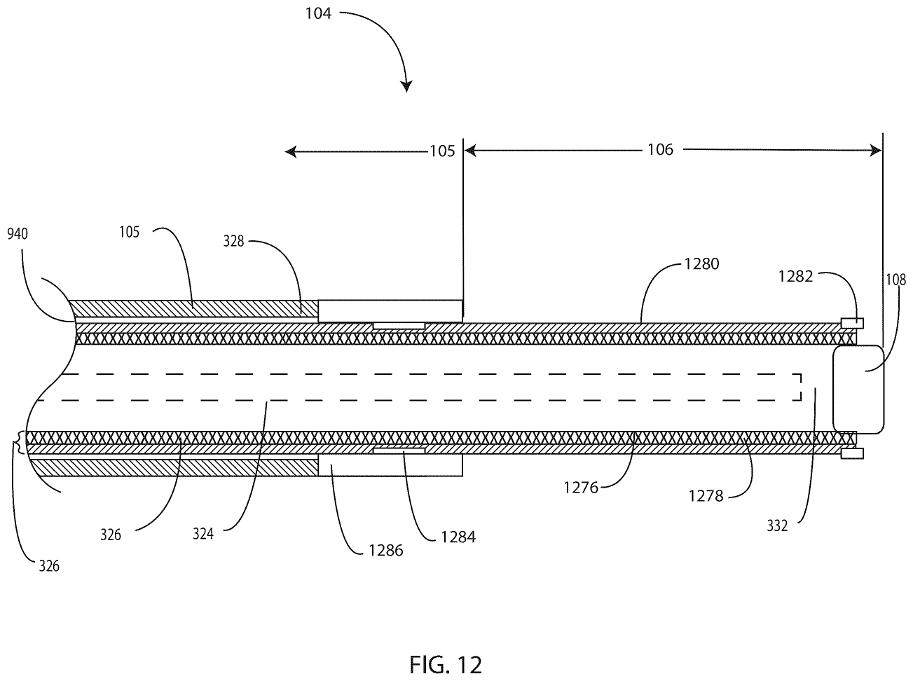

### Continuous Return Tube (FIG. 12)

Referring now to FIG. 12, a schematic view of a portion of a cryoablation shaft is shown in accordance with various embodiments herein. In various embodiments, the shaft includes an insulated zone 105 and an expansion chamber 106. In various embodiments, the insulated zone 105 of shaft 104 includes a supply tube 324 located within a return tube 326. In various embodiments, after exiting the handle 102, the high-pressure flow of the working fluid travels down the supply tube 324. The high-pressure stream of the working fluid may undergo expansion at or downstream of the Joule-Thomson orifice 332 and returns down the shaft in the annular space between the supply tube 324 and the return tube 326.

In various embodiments, the return tube 326 can continuously span the entire length of the shaft 104. For instance, the return tube 326 can be a unitary component spanning throughout the insulated zone 105 the expansion chamber 106 of the shaft 104. In various embodiments, the return tube 326 can be a composite shaft formed of two or more layers. In the example of FIG. 12, the return tube 326 can include a first polymer layer 1276, a reinforcement layer 1278, and a second polymer layer 1280.

In various embodiments, the first polymer layer 1276 forms the innermost layer of the return tube 326. In various embodiments, the first polymer layer 1276 is configured to contain fluid from the working fluid circuit. In particular, the first polymer layer 1276 is configured to contain the working fluid returning to the handle in the annular space between the supply tube 324 and the return tube 326.

The first polymer layer 1276 can be formed from any suitable polymer or blend of polymers such as polyethylene terephthalate (PET), PTFE, ePTFE, PEEK, polyetherimide (PEI), polyimide (PI), or the like. In various embodiments, the first polymer layer 1276 is gas impermeable.

In various embodiments, the return tube 326 can include a reinforcement layer 1278 surrounding the first polymer layer 1276. In various embodiments, the reinforcement layer 1278 is configured to increase the radial strength of the composite shaft. The reinforcement layer 1278 may also enhance additional functional properties of the shaft 104 such as stiffness, burst pressure resistance, torque transmission, kink resistance, or the like. For instance, the reinforcement layer 1278 can be configured to improve the ability of the first polymer layer 1276 and/or the second polymer layer 1280 to traverse turns and corners without kinking or other damage.

In some embodiments, the reinforcement layer 1278 can be formed from a metallic material such as Stainless Steel, Titanium, Tungsten, or the like. In some embodiments, the reinforcement layer 1278 can be formed from a polymeric material such as Kevlar, polyimide, PET, or the like.

In various embodiments, the reinforcement layer 1278 can be formed from a braided material having any suitable combination of braid pattern, pick count (measured in picks per inch, or ppi), material, wire dimension, wire size/shape, durometer, or the like as described in detail in the description of FIG. 9. In the example of FIG. 12, the reinforcement layer 1278 has a uniform braid density along the entire length of the return tube 326. The braid density of reinforcement layer 1278 can be selected to enhance the radial strength of the shaft while preserving the ability of the shaft to navigate turns and corners to access a cryoablation site. In an embodiment, the shaft 104 may be constructed to have a radius of curvature of less than or equal to 30 mm. In alternate embodiments, the reinforcement layer 1278 can have any suitable density profile along the length of the shaft 104. In alternative embodiments, the reinforcement layer 1278 can be formed from one or more coils such as described in detail in the description of FIG. 9.

In various embodiments, the return tube 326 can include a second polymer layer 1280 surrounding the reinforcement layer 1278 and the first polymer layer 1276. The second polymer layer 1280 can be formed from any suitable polymer or polymers including but not limited to, thermoplastic polymers, biocompatible polymers, polyether block amides such as Pebax, or the like. In various embodiments, at least one of the first polymer layer 1276 and the second polymer layer 1278 is impermeable.

The second polymer layer 1280 can serve a variety of functions. In various embodiments, the second polymer layer 1280 functions as a redundant gas containment layer to the first polymer layer 1276. In various embodiments, the second polymer layer 1280 is configured to provide additional strength and reinforcement to the first polymer layer 1276 and the reinforcement layer 1278, thus reducing the likelihood of any leak or rupture in the first polymer layer.

In various embodiments, the second polymer layer 1280 is formed from a biocompatible material. A biocompatible material, as defined herein, is any material that is compatible with living tissues or organisms without causing harm or adverse reactions. In various embodiments, forming the outermost layer of the shaft 104 from a biocompatible material minimizes negative patient outcomes such as inflammation, toxicity, or rejection in a patient while performing a cryoablation procedure.

In various embodiments, the second polymer layer 1280 is formed from a material having radiopaque properties. In various embodiments, radiopaque materials are not visible under radiation. In an embodiment, the second polymer layer 1280 can be fortified with a radiopaque material, such as tungsten, to improve the visibility of the expansion chamber under imaging procedures such as fluoroscopy.

In various embodiments, the second polymer layer 1280 is configured to exhibit stability over the full range of cryoablation operating temperatures and pressures. In various embodiments, the second polymer layer 1280 is configured to exhibit low dimensional changes over the full range of cryoablation temperatures. Cryoablation operating temperatures range from body temperature of a patient (approximately 310 Kelvin) to cryoablation temperatures (approximately 190 Kelvin), and the device can undergo temperature transitions over this range. For instance, the second polymer layer may experience little to no dimensional changes going from either room temperature (approximately 293 Kelvin) or the body temperature of a patient (approximately 310 Kelvin) to cryoablation temperatures (approximately 190 Kelvin). In various embodiments, the second polymer layer 1280 is configured to exhibit low dimensional changes over the full range of cryoablation pressures. For instance, the second polymer layer may experience little to no dimensional changes going from vacuum pressure (approximately 0.05 Torr) to high-pressures associated with the Joule-Thomson orifice (between about 6.9 MPa and about 13.8 MPa, e.g., about 12.4 MPa).

In some embodiments, the outer diameter of the second polymer layer 1280 experiences a percent change of less than or equal to 5%, 4%, 3%, 2%, 1%, or 0%, from its original diameter over the course of a cryoablation procedure. In some embodiments, the length of the second polymer layer 1280 experiences a percent change of less than or equal to 5%, 4%, 3%, 2%, 1%, or 0%, from its original length over the course of a cryoablation procedure.

In various embodiments, the second polymer layer 1280 is configured to maintain a high burst strength over the full range of cryoablation pressures and temperatures. In some embodiments, the burst strength of the second polymer layer 1280 can be greater than or equal to 12.4 MPa, 13.1 MPa, 13.8 MPa, 14.5 MPa, 15.2 MPa, 27.6 MPa, or 41.4 MPa. In some embodiments, the burst strength of each of the first second polymer layer 1280 can be less than or equal to 41.4 MPa, 20.7 MPa, 19.3 MPa, 17.9 MPa, 16.5 MPa, or 15.2 MPa. In some embodiments, the burst strength can fall within a range of 12.4 MPa to 41.4 MPa, or 13.1 MPa to 27.6 MPa, or 13.8 MPa to 17.9 MPa, or 14.5 MPa to 16.5 MPa, or can be about 15.2 MPa.

In some embodiments, the burst strength of the second polymer layer 1280 can be greater than or equal to 0.25 MPa, 0.62 MPa, or 1.00 MPa. In some embodiments, the burst strength of the second polymer layer 1280 can be less than or equal to 4.00 MPa, 2.50 MPa, or 1.00 MPa. In some embodiments, the burst strength of the second polymer layer 1280 can fall within a range of 0.25 MPa to 4.00 MPa, or 0.62 MPa to 2.50 MPa, or can be about 1.00 MPa.

In various embodiments, the second polymer layer 1280 is configured to maintain its elasticity over the full range of cryoablation pressures and temperatures. For instance, second polymer layer 1280 can facilitate the shaft 104 being sufficiently flexible, such that the shaft may form a curve having the smallest radius of curvature of less than or equal to 30 mm, 20 mm, 10 mm, or 5 mm over the full range of cryoablation pressures and temperatures.

In various embodiments, the return tube 326 can be sealingly joined to the insulating shaft 328 by any suitable method or methods. In the example of FIG. 12 the return tube 326 is sealed to the insulating shaft 328 using a reflow process. In particular, the second polymer layer 1280 of the composite shaft is sealed to the insulating shaft using a thermal reflow processes. Thermal reflow refers to the softening and material displacement that occurs in polymers when heated above their glass transition temperature. In such embodiments it desirable for the second polymer layer 1280 to be formed from a material or materials that can be repeatedly melted without effecting its material properties such as thermoplastics or the like.

To form the connection between the return tube 326 and the insulating shaft 328 as seen in FIG. 12, the return tube 326 can be slid into the insulating shaft 328. In various embodiments, the return tube 326 can be slid into the insulating shaft 328 by a predetermined amount to set the length of the expansion chamber 106. As detailed in the description FIG. 6, the length of the expansion chamber 106 affects the size and shape of the ice balls formed by the cryoablation probe.

In some embodiments, a first piece of tubing 1284 can be slid between the inner shaft and outer shaft. In various embodiment, the first piece of tubing 1284 can be any suitable material or materials such as Pebax, or the like. In various embodiments, the first piece of tubing 1284 can have a lower glass transition temperature than the other components of the shaft 104. The shaft 104 can then be subject to sufficiently high temperatures to melt the first piece of tubing 1284 in a reflow process.

In some embodiments, a second piece of tubing 1286 is slid onto the shaft 104 over the return tube 326, insulating shaft 328, and first piece of tubing 1284. In various embodiments, the second piece of tubing 1286 can be any suitable material or materials such as Pebax, or the like. The shaft 104 can then be subject to sufficiently high temperatures to melt the first piece of tubing 1284 and/or the second piece of tubing 1286 in a reflow process. In various embodiments, the shaft is subject to compressive forces during the reflow process, resulting in a leak tight seal between the return tube 326 and the insulating shaft 328.

In alternate embodiments, the insulating shaft 328 can be sealed to the outer layer of the return tube 326 by any suitable means such as adhesives, or the like. For instance, an adhesive can be applied between the inner layer insulating shaft 328 and the outer layer of the return tube 326. The adhesive should be selected to have suitable material properties to adhere to both the insulating shaft 328 and the return tube 326 over the entire range of cryoablation operating temperatures and pressures.

In various embodiments, the return tube 326 can be sealingly joined to distal operating tip 108 by any suitable method or methods. In the example of FIG. 12 the return tube 326 is sealed to the insulating shaft 328 using a swaging process. To form the connection between the return tube 326 and the distal operating tip 108 as seen in FIG. 12, a joining element such as a swage ring 1282, or the like is loaded onto the distal end of the return tube 326. The distal operating tip 108 can then be inserted into the return tube. The shaft 104 can then be inserted into a piece of swaging equipment having one or more swage dies. The swaging equipment applies a rotating radial force to the swage ring 1282, causing the diameter of the swage ring to decrease. This reduction in diameter compresses the return tube 326 between the swage ring 1282 and distal operating tip 108. Such a process forms a leak tight seal between the return tube 326 and the distal operating tip 108. In the embodiment of FIG. 12, the second polymer layer 1280 is formed from a continuous tube of material. The second polymer layer can have substantially uniform material properties (e.g., flexibility burst strength, kink resistance, hardness, gas permeability, visibility) along the entire length of the shaft 104. In a particular embodiment, the second polymer layer 1280 has an approximately constant durometer of hardness along its length. In some embodiments, the hardness of the second polymer layer 1280 can be greater than or equal to 50, 57, 63, or 70 Shore A. In some embodiments, the hardness of the second polymer layer 1280 can be less than or equal to 100, 90, 80, or 70 Shore A. In some embodiments, the hardness of the second polymer layer 1280 can fall within a range of 50 to 100 Shore A, or 57 to 90 Shore A, or 63 to 80 Shore A, or can be about 70 Shore A. However, in alternative embodiments, the second polymer layer 1280 may be configured to have variable material properties along the length of the shaft 104.

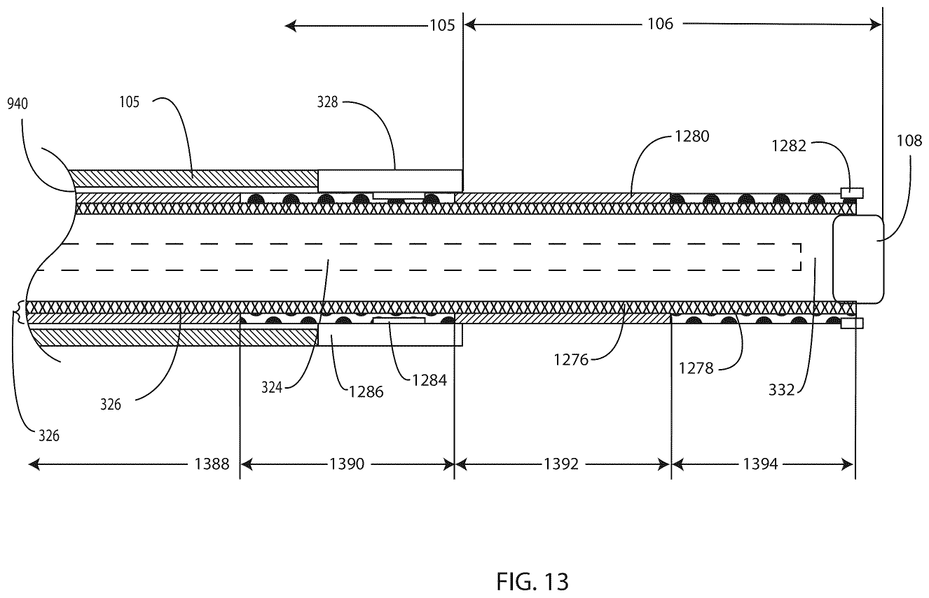

### Continuous Return Tube—Multi-Zone Embodiment (FIG. 13)

Referring now to FIG. 13, a schematic view of a portion of a cryoablation shaft is shown in accordance with various embodiments herein. The cryoablation shaft 104 of FIG. 13 is substantially similar to the cryoablation shaft 104 of FIG. 12 except that the second polymer layer 1280 is configured to have variable material properties along the length of the shaft 104.

In various embodiments, the second polymer layer 1280 is formed from two or more zones, wherein each zone can be formed from a discrete segment of polymer tubing. Each of the tubing segments can be formed from any suitable thermoplastic material such as Pebax, or the like. In the example of FIG. 13, the second polymer layer 1280 is formed from four zones 1388, 1390, 1392, 1394.

In various embodiments, the discrete zones can be formed into the second polymer layer 1280 of the return tube 326 by any suitable method or methods. In the example of FIG. 13 the second polymer layer 1280 is formed using a thermal reflow process. The desired number of discrete tube segments (in this case four discrete tube segments corresponding to the four zones 1388, 1390, 1392, 1394) are loaded onto the shaft 104 over the outermost layer of the return tube 326 (in this case the reinforcement layer 1278). The discrete tube segments are then fused to each other and to the outermost layer of the return tube 326 by subjecting the shaft to heat and compressive forces in a thermal reflow process.

In the example of FIG. 13, the second polymer layer 1280 includes four discrete tube segments corresponding to four zones 1388, 1390, 1392, 1394. However, the second polymer layer 1280 may include any suitable number of zones such as one, three, five, or more zones. In the example of FIG. 13, each of the zones 1388, 1390, 1392, 1394 are of approximately similar length. However, in alterative embodiments, each of the zones can vary in length based on the selection of tube segments.

In various embodiments, each of the zones of the second polymer layer 1280 can have a set of material properties (e.g., flexibility burst strength, hardness, kink resistance, gas permeability, visibility) selected to enhance various performance parameter of the shaft 104. In various embodiments, each of the discrete tube segments can have the same or different material properties depending on the desired performance parameters of the shaft (e.g., strength, flexibility, biocompatibility, durability, or the like).

In one example, the distal operating tip 108 being inserted into the shaft 104 can reduce the flexibility of the shaft at its distal end. In such circumstances, it may be advantageous to tailor the second polymer layer 1280 to increase in flexibility (or decrease in hardness) towards the distal tip 108 of the shaft 104.

In various embodiments, the second polymer layer 1280 is a thermoplastic material having a plurality of zones having different durometers of hardness along its length. In some embodiments, the second polymer layer 1280 includes at least one decrease in hardness from a proximal end of the shaft towards a distal end of the shaft.

In various embodiments the second polymer layer 1280 can include at least a first zone having a first durometer of hardness and a second zone distal to the first zone having a second durometer of hardness. In some embodiments, the first durometer of hardness can be greater than or equal to 60, 63, 67, or 70 Shore A. In some embodiments, the first durometer of hardness can be less than or equal to 80, 77, 73, or 70 Shore A. In some embodiments, the first durometer of hardness can fall within a range of 60 to 80 Shore A, or 63 to 77 Shore A, or 67 to 73 Shore A, or can be about 70 Shore A. In some embodiments, the second durometer of hardness can be greater than or equal to 35, 42, 48, or 55 Shore A. In some embodiments, the second durometer of hardness can be less than or equal to 75, 68, 62, or 55 Shore A. In some embodiments, the second durometer of hardness can fall within a range of 35 to 75 Shore A, or 42 to 68 Shore A, or 48 to 62 Shore A, or can be about 55 Shore A.

In the example of FIG. 13, the second polymer layer 1280 includes a first zone 1388, second zone 1390, third zone 1392, and fourth zone 1394. Each of the zones can have the same or different material properties as their adjacent zones. In an embodiment, the first zone 1388 can have a first durometer of hardness, the second zone 1390 can have a second durometer of hardness, the third zone 1392 can have a third durometer of hardness, and the fourth zone 1394 can have a fourth durometer of hardness. In one particular example, the hardness of each zone can decrease from the first zone 1388 to the fourth zone. However, the hardness profile of the second polymer layer 1280 can vary based on the configuration of the shaft 104 and its intended use.

In various embodiments, the various zones of the second polymer layer 1280 can be tailored to improve the performance of the shaft. For instance, particular zones can be fortified with one or more materials. In an embodiment, one or more of the zones of the second polymer layer 1280 can be fortified with a radiopaque material, such as tungsten to improve the visibility of the shaft 104 under fluoroscopy.

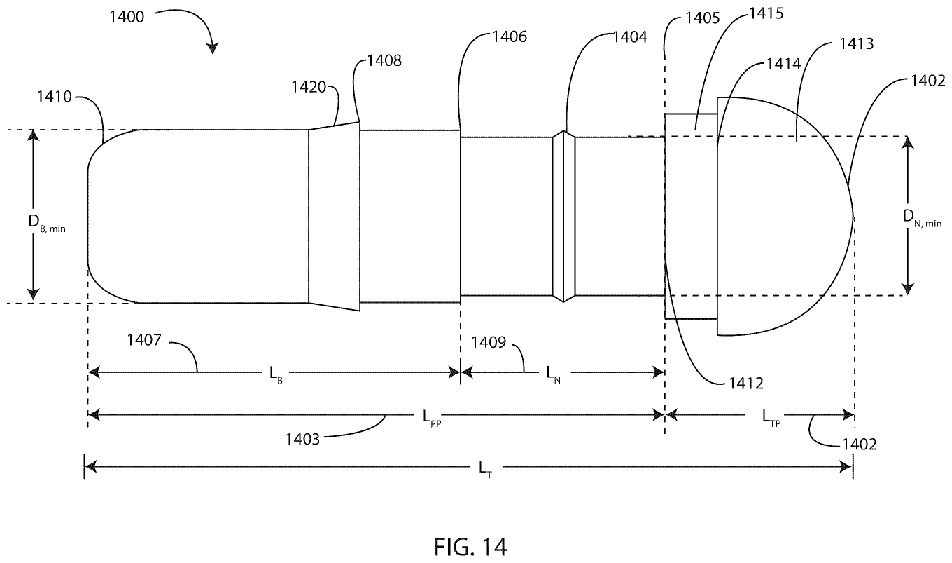

Distal Tip with Swage Ring (FIGS. 14-16)

Referring now to FIG. 14, a schematic view of a distal tip is shown in accordance with various embodiments herein. In various embodiments, the distal tip 1400 is configured to seal a distal end of the return tube 328 of a cryoablation shaft 104 when the shaft 104 is compressed around the distal tip using a compressive element such as a ring. In various embodiments, the distal tip is configured to maintain a vacuum tight seal of the shaft over the full range of cryoablation temperatures and pressures. The distal tip 1400 can be formed from any suitable material or materials including metals such as stainless steel, nitinol, or the like.

In various embodiments, the distal tip 1400 can have a total length LT. In some embodiments, the length LT can be greater than or equal to 2.0 mm, 3.2 mm, 4.5 mm, or 5.7 mm. In some embodiments, the length LT can be less than or equal to 10.0 mm, 8.6 mm, 7.1 mm, or 5.7 mm. In some embodiments, the length LT can fall within a range of 2.0 mm to 10.0 mm, or 3.2 mm to 8.6 mm, or 4.5 mm to 7.1 mm, or can be about 5.7 mm.

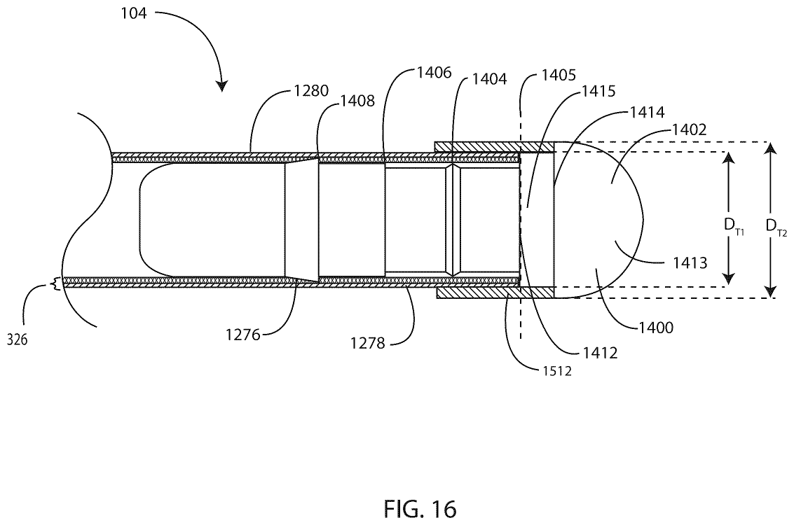

In various embodiments the distal tip 1400 can include a tip portion 1402. The tip portion 1402 is configured to extend distally from the distal end of the return tube 326 when the distal tip 1400 is inserted into a cryoablation shaft 104 (such as depicted by FIG. 16). In the example of FIG. 14, the tip portion 1402 forms an atraumatic surface which is rounded and smooth. Atraumatic tips are configured to guide the cryoablation shaft 104 through a patient's body while avoiding perforation or other damage to the patient's anatomy. Such a configuration is useful in scenarios such as ablating lesions in the biliary system where it is generally undesirable to pierce the patient's tissue. In alternate configurations, the tip portion 1402 may include a sharp tip, such as a trocar tip, or the like.

In various embodiments the tip portion 1402 can include one or more features to facilitate the attachment of a flexible polymeric tip. In various embodiments, the flexible polymeric tip can have a tapered profile to facilitate crossing of lesions. In various embodiments, the flexible polymeric tip can incorporate a guidewire lumen configured for the insertion of a guidewire.

In various embodiments, the tip portion 1402 can have a length L_{TP}. In some embodiments, the length L_{TP} can be greater than or equal to 0.5 mm, 0.8 mm, 1.1 mm, or 1.4 mm. In some embodiments, the length L_{TP} can be less than or equal to 3.0 mm, 2.5 mm, 1.9 mm, or 1.4 mm. In some embodiments, the length L_{TP} can fall within a range of 0.5 mm to 3.0 mm, or 0.8 mm to 2.5 mm, or 1.1 mm to 1.9 mm, or can be about 1.4 mm. In various embodiments, the tip portion 1402 can include a first tip section 1413 and a second tip section 1415 proximal to the first tip section. In the example of FIG. 14, the first tip section 1413 can be substantially hemispherical in profile and the second tip section 1415 can be substantially cylindrical in profile. In various embodiments a proximal end of the first tip section 1413 has a larger diameter than a distal end of the second tip section 1415. The tip portion can further include a ledge 1414 between the first tip section 1413 and the second tip section 1415.

In various embodiments the distal tip 1400 can include a plug portion 1403 configured to be inserted inside of the return tube 326 when the distal tip 1400 is inserted into a cryoablation shaft 104, such as depicted by FIG. 16. FIG. 16 shows a plane 1405 designating the boundary between the tip portion 1402 and the plug portion 1403. In various embodiments, the distal tip 1400 is configured to be inserted into the distal end of a shaft 104 up to the plane 1405.

In various embodiments the plug portion 1403 can have a length L_{PP}. In some embodiments, the length L_{PP} can be greater than or equal to 1.0 mm, 2.1 mm, 3.2 mm, or 4.3 mm. In some embodiments, the length L_{PP} can be less than or equal to 9.0 mm, 7.4 mm, 5.9 mm, or 4.3 mm. In some embodiments, the length L_{PP} can fall within a range of 1.0 mm to 9.0 mm, or 2.1 mm to 7.4 mm, or 3.2 mm to 5.9 mm, or can be about 4.3 mm.

In various embodiments, the length L_{PP} of the plug portion 1403 is greater than the length L_{TP} of the tip portion 1402. In some scenarios, having a plug portion 1403 that is longer than the tip portion 1402 can provide increased support to the distal tip 1400, increasing the strength of the connection to the shaft 104. In some embodiments, the ratio of the lengths L_{PP}/L_{TP} can be greater than or equal to 1.5, 2, 2.5, or 3. In some embodiments, the ratio of the lengths L_{PP}/L_{TP} can be less than or equal to 5, 4.3, 3.7, or 3. In some embodiments, the ratio of the lengths L_{PP}/L_{TP} can fall within a range of 1.5 to 5, or 2 to 4.3, or 2.5 to 3.7, or can be about 3.

In various embodiments, at least a portion of the plug portion 1403 has a diameter that is larger than an inner diameter of the return tube 326. Such a configuration creates an interference fit between the distal tip 1400 and the return tube 326, forming a robust mechanical joint and gas tight seal. In some embodiments, the difference between the outer diameter of at least a portion of the plug portion 1403 and the inner diameter of the return tube 326 can be at least 0.01 mm, 0.06 mm, 0.11 mm, 0.16 mm, 0.21 mm, or 0.25 mm or larger.

In various embodiments, a proximal end of the tip portion 1402 has a larger diameter than a distal end of the plug portion 1403. The distal tip 1400 can further include a tip shoulder 1412 between the tip portion 1402 and the plug portion 1403. In various embodiments, a distal end of the return tube 326 is configured to abut the distal tip 1400 at the tip shoulder 1412 when the distal tip is inserted into the shaft 104.

In various embodiments the plug portion 1403 can include a base section 1407 and a neck section 1409. The base section 1407 is configured to be proximal to the neck section 1409 when the distal tip 1400 is inserted into a cryoablation shaft 104.

In various embodiments the base section 1407 can have a length L_{B}. In some embodiments, the length L_{B} can be greater than or equal to 1.0 mm, 1.6 mm, 2.2 mm, or 2.8 mm. In some embodiments, the length L_{B} can be less than or equal to 5.0 mm, 4.3 mm, 3.5 mm, or 2.8 mm. In some embodiments, the length L_{B} can fall within a range of 1.0 mm to 5.0 mm, or 1.6 mm to 4.3 mm, or 2.2 mm to 3.5 mm, or can be about 2.8 mm.

In various embodiments, the base section 1407 has a minimum base diameter D_{B, min}. Along the length of the base section 1407, the diameter of the base section 1407 is not smaller than the minimum base diameter. In various embodiments, the minimum base diameter D_{B, min} is approximately equal to the inner diameter of the return tube 326. Alternatively, the minimum base diameter D_{B}, min can be marginally smaller or marginally larger than the inner diameter of the return tube 326. In some embodiments, the minimum base diameter can be greater than or equal to 0.8 mm, 1.0 mm, 1.2 mm, or 1.3 mm. In some embodiments, the minimum base diameter can be less than or equal to 2.5 mm, 2.1 mm, 1.8 mm, or 1.3 mm. In some embodiments, the minimum base diameter can fall within a range of 0.8 mm to 2.5 mm, or 1.0 mm to 2.1 mm, or 1.2 mm to 1.8 mm, or can be about 1.3 mm.

In various embodiments the neck section 1409 can have a length LN. In some embodiments, the length LN can be greater than or equal to 0.5 mm, 0.8 mm, 1.2 mm, or 1.5 mm. In some embodiments, the length LN can be less than or equal to 3.0 mm, 2.5 mm, 2.0 mm, or 1.5 mm. In some embodiments, the length LN can fall within a range of 0.5 mm to 3.0 mm, or 0.8 mm to 2.5 mm, or 1.2 mm to 2.0 mm, or can be about 1.5 mm.

In various embodiments, the neck section 1409 has a minimum neck diameter D_{N, min} long the length of the neck section 1409, the diameter of the neck section is not smaller than the minimum neck diameter. In various embodiments, minimum neck diameter D_{N, min} is smaller than the inner diameter of the return tube 326. In some embodiments, the difference between the inner diameter of the return tube 326 and the minimum neck diameter can be at least 0.01 mm, 0.06 mm, 0.11 mm, 0.16 mm, 0.21 mm, or 0.25 mm or larger. Alternatively, the minimum neck diameter D_{N, min} can be approximately equal to or marginally larger than the inner diameter of the return tube 326.

In some embodiments, the minimum neck diameter D_{N, min} can be greater than or equal to 0.5 mm, 0.7 mm, 1.0 mm, or 1.2 mm. In some embodiments, the minimum neck diameter D_{N, min} can be less than or equal to 2.0 mm, 1.7 mm, 1.5 mm, or 1.2 mm. In some embodiments, the minimum neck diameter D_{N, min} can fall within a range of 0.5 mm to 2.0 mm, or 0.7 mm to 1.7 mm, or 1.0 mm to 1.5 mm, or can be about 1.2 mm.

In various embodiments the distal tip 1400 can include a plug shoulder 1406 between the neck section 1409 and the base section 1407 of the plug portion 1403. In the example of FIG. 14, the plug shoulder spans the difference in diameter between the minimum base diameter D_{B}, min and the minimum neck diameter D_{N, min}.

In various embodiments the distal tip 1400 can include a barb 1408 on the base section 1407. The barb 1408 has a diameter along its length that is larger than the minimum base diameter. In various embodiments, the barb 1408 includes a ramp 1420 leading up to the largest an outer diameter location. The ramp 1420 facilitates sliding the distal tip 1400 into the shaft 104. In various embodiments, the barb 1408 is configured to form an interference fit with the return tube 326 when the distal tip is inserted into the shaft 104. In some embodiments, the difference between the outer diameter of the barb 1408 and the inner diameter of the return tube 326 can be at least 0.01 mm, 0.06 mm, 0.11 mm, 0.16 mm, 0.21 mm, or 0.25 mm or larger. Such a configuration creates a robust mechanical joint and gas tight seal between the return tube 326 and the distal tip 1400.

In the example of FIG. 14, the distal tip 1400 includes one barb 1408. However, the distal tip 1400 may include two, three, or more barbs 1408 disposed along the length of the plug portion 1403.

In various embodiments the distal tip 1400 can include a protrusion 1404 on the neck section 1409. In various embodiments, the protrusion 1404 is configured to form an interference fit with the return tube 326 when the distal tip is inserted into the shaft 104. In some embodiments, the difference between the outer diameter of the protrusion 1404 and the inner diameter of the return tube 326 can be at least 0.01 mm, 0.06 mm, 0.11 mm, 0.16 mm, 0.21 mm, or 0.25 mm or larger. Such a configuration creates a robust mechanical joint and gas tight seal between the return tube 326 and the distal tip 1400.

In the example of FIG. 14, the protrusion 1404 converges to a single point. However, the protrusion 1404 can be of any suitable profile configured to grab onto the inner surface of the return tube 326. In the example of FIG. 14, the distal tip 1400 includes one protrusion 1404. However, the distal tip may include two, three, or more protrusions disposed along the length of the plug portion 1403.

In various embodiments, the proximal end of the distal tip 1400 comprises one or more curved edges 1410. In various embodiments, the curved edges 1410 are configured to prevent the distal tip 1400 from damaging the shaft 104 as the shaft navigates turns and corners during a cryoablation procedure. In various embodiments, the curved edges 1410 are configured to prevent the distal tip 1400 from damaging the return tube 326 while inserting and navigating the cryoablation shaft to a treatment site.

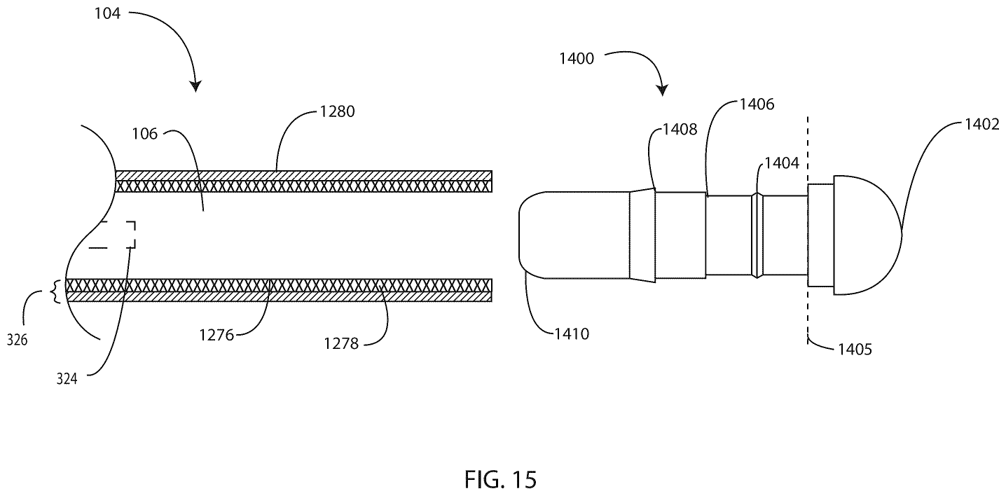

Referring now to FIG. 15, an exploded view of a distal tip and the distal end of a cryoablation shaft is shown in accordance with various embodiments herein. Referring now to FIG. 16, a schematic view of a distal end of a cryoablation shaft sealed with a distal tip is shown in accordance with various embodiments herein. In various embodiments, the distal tip 1400 is configured to seal a distal end of the return tube 328 of a cryoablation shaft 104.

In various embodiments, the return tube 326 can be sealingly joined to the distal tip 1400 by any suitable method or methods. In the example of FIGS. 15-16, the return tube 326 is sealed to the distal tip 1400 using a swaging process. To form the connection between the return tube 326 and the distal tip 1400, as seen in FIG. 16, a compressive element such as a ring 1512, or, more particularly, a swage ring, or the like, is loaded onto the distal end of the return tube 326. The distal tip 1400 can then be inserted into the return tube 326.

The shaft 104 can then be inserted into a piece of swaging equipment having one or more swage dies. The swaging equipment applies a radial force to the ring 1512, causing the diameter of the swage ring to decrease. This reduction in diameter compresses the return tube 326 between the swage ring 1512 and distal tip 1400. Such a process forms a leak tight seal between the return tube 326 and the distal tip 1400 and increases the burst strength of distal tip to shaft joint. In some embodiments, the radial force is a rotating radial force.

In various embodiments, the distal tip 1400 is configured to be inserted into the distal end of a shaft 104 up to the plane 1405. When the distal tip is inserted into the shaft 104, as best seen in FIG. 16, a distal end of the return tube 326 is configured to abut the distal tip 1400 at the tip shoulder 1412. In alternate configurations a distal end of the return tube 326 is configured to abut the distal tip 1400 proximal to the tip shoulder 1412. In either configuration, the tip shoulder 1412 is configured to prevent the distal tip 1400 from slipping into the return tube 326 in the event that the distal tip starts to slip with respect to the return tube.

In various embodiments, a compressive element, such as ring 1512 is configured to surround the return tube 326 and the plug portion 1403 of the distal tip 1400, sealing the distal tip to the return tube. In various embodiments, the ring 1512 is configured to apply a consistent compressive force around the return tube 326, sealing the distal tip 1400 to the return tube 326.

When the distal tip is inserted into the shaft 104, as best seen in FIG. 16, a distal end of ring 1512 is configured to abut the distal tip 1400 at the ledge 1414. In alternate configurations a distal end of the return tube 326 is configured to abut the distal tip 1400 proximally to the to the ledge 1414 (e.g., at the tip shoulder 1412). In either configuration, the ledge 1414 is configured to prevent the ring 1512 from slipping off of the distal tip 1400 in the event that the ring starts to slip with respect to the distal tip.

In the embodiment of FIG. 16, the ring 1512 terminates distally to the return tube 326 along the length of the distal tip 1400. In alternate configurations, the ring 1512 terminates at the same location as the return tube 326 along the length of the distal tip 1400. For instance, both the ring 1512 and the return tube can terminate at tip shoulder 1412.

In various embodiments, the protrusion 1404 disposed on the neck section 1409 of the distal tip 1400 is configured to form a seal with the return tube 326 when the distal tip 1400 is inserted into the shaft 104. In some embodiments, the ring 1512 is configured to seal to the return tube 326 around the protrusion 1404. In such embodiments, the ring 1512 is configured to apply a compressive force on the return tube 326, pressing the return tube into the protrusion 1404, which creates a fluid-tight seal between the distal tip 1400 and the return tube.

In various embodiments, the plug shoulder 1406 disposed between the neck section 1409 and the base section 1407 of the distal tip 1400 is configured to create a hard stop for the ring 1512. In the event the distal plug moves relative to the shaft 104, the swage ring will stop on the plug shoulder 1406 and prevent further movement.

In various embodiments, the tip portion 1402 can have a first tip diameter D_{T1} corresponding to the diameter of the second tip section 1415. In some embodiments, the first tip diameter D_{T1} can be greater than or equal to 1 mm, 1.1 mm, 1.2 mm, 1.3 mm, or 1.4 mm. In some embodiments, the first tip diameter D_{T1} can be less than or equal to 1.8 mm, 1.7 mm, 1.6 mm, 1.5 mm, or 1.4 mm. In some embodiments, first tip diameter D_{T1} can fall within a range of 1.0 mm to 1.8 mm, or 1.1 mm to 1.7 mm, or 1.2 mm to 1.6 mm, or 1.3 mm to 1.5 mm, or can be about 1.4 mm. In various embodiments, the first tip diameter D_{T1} is approximately equal to the outer diameter of the return tube 326. Such a configuration creates a smooth transition between the return tube 326 and the distal tip 1400.

In various embodiments, the tip portion 1402 can have a second tip diameter D_{T2} corresponding to the diameter of the first tip section 1413 at the ledge 1414. In some embodiments, the second tip diameter D_{T2} can be greater than or equal to 1.2 mm, 1.4 mm, 1.6 mm, or 1.8 mm. In some embodiments, the second tip diameter D_{T2} can be less than or equal to 2.5 mm, 2.3 mm, 2.0 mm, or 1.8 mm. In some embodiments, the second tip diameter D_{T2} can fall within a range of 1.2 mm to 2.5 mm, or 1.4 mm to 2.3 mm, or 1.6 mm to 2.0 mm, or can be about 1.8 mm. In various embodiments, the second tip diameter D_{T2} is approximately equal to the outer diameter of the ring 1512. Such a configuration creates a smooth transition between the ring 1512 and the distal tip 1400.

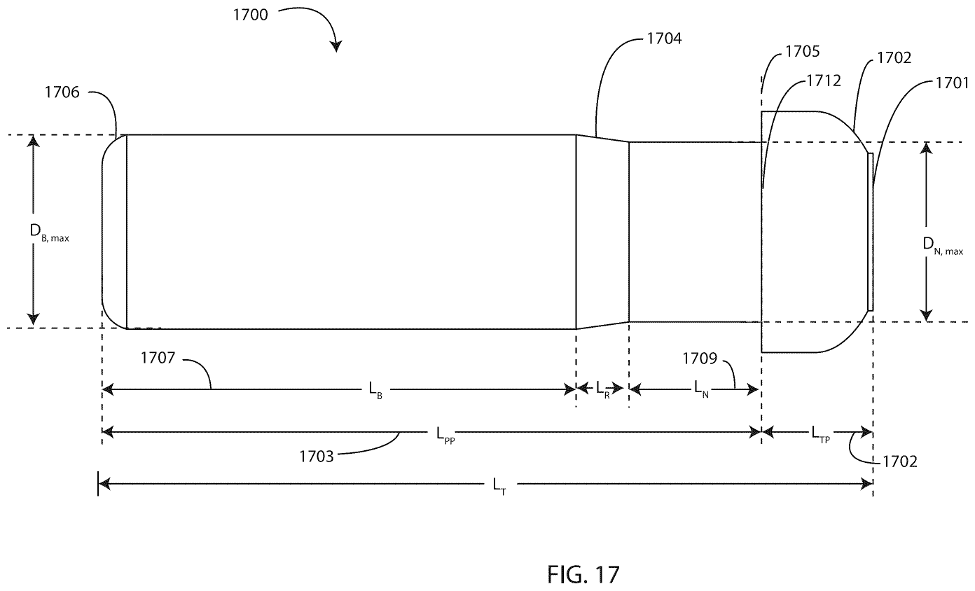

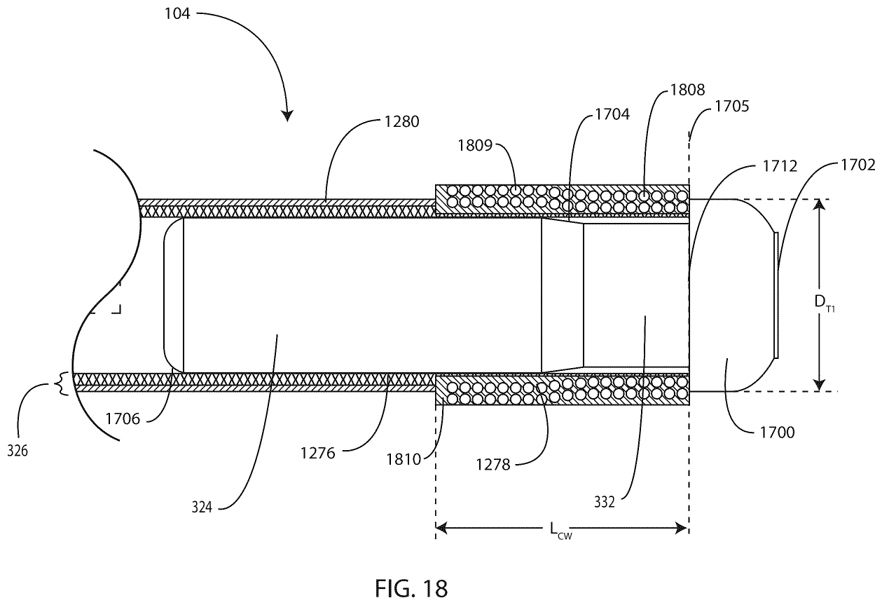

Distal Tip with Compressive Wrap (FIGS. 17-18)

Referring now to FIG. 17, a schematic view of a distal tip is shown in accordance with various embodiments herein. In various embodiments, the distal tip 1700 is configured to seal a distal end of the return tube 328 of a cryoablation shaft 104 when the shaft 104 is compressed around the distal tip using a compressive wrap. The distal tip 1700 can be formed from any suitable material or materials including metals such as stainless steel, nitinol, or the like.

In various embodiments, the tip portion can have a total length LT. In some embodiments, the length LT can be greater than or equal to 2.0 mm, 3.2 mm, 4.5 mm, or 5.7 mm. In some embodiments, the length LT can be less than or equal to 10.0 mm, 8.6 mm, 7.1 mm, or 5.7 mm. In some embodiments, the length LT can fall within a range of 2.0 mm to 10.0 mm, or 3.2 mm to 8.6 mm, or 4.5 mm to 7.1 mm, or can be about 5.7 mm.

In various embodiments the distal tip 1700 can include a tip portion 1702. The tip portion 1702 is configured to extend distally from the distal end of the return tube 326 when the distal tip 1700 is inserted into a cryoablation shaft 104 (such as depicted by FIG. 18). In the example of FIG. 17, the tip portion 1702 forms an atraumatic surface which is rounded and smooth at the outside edges. In alternate configurations, the tip portion 1702 may have a hemispherical distal surface like the tip portion 1402 or may include a sharp tip, such as a trocar tip, or the like.

In various embodiments the tip portion 1702 can have a length L_{TP}. In some embodiments, the length L_{TP} can be greater than or equal to 0.5 mm, 0.6 mm, 0.8 mm, or 0.9 mm. In some embodiments, the length L_{TP} can be less than or equal to 2.0 mm, 1.6 mm, 1.3 mm, or 0.9 mm. In some embodiments, the length L_{TP} can fall within a range of 0.5 mm to 2.0 mm, or 0.6 mm to 1.6 mm, or 0.8 mm to 1.3 mm, or can be about 0.9 mm.

In various embodiments, the distal tip 1700 can include a plug portion 1703 configured to be inserted inside of the return tube 326 when the distal tip 1700 is inserted into a cryoablation shaft 104, such as depicted by FIG. 18. FIG. 17 shows a plane 1705 designating the boundary between the tip portion 1702 and the plug portion 1703. In various embodiments, the distal tip 1700 is configured to be inserted into the distal end of a shaft 104 up to the plane 1705.

In various embodiments the plug portion 1703 can have a length L_{PP}. In some embodiments, the length L_{PP} can be greater than or equal to 2.5 mm, 3.2 mm, 3.9 mm, or 4.8 mm. In some embodiments, the length L_{PP} can be less than or equal to 8.0 mm, 6.9 mm, 5.7 mm, or 4.8 mm. In some embodiments, the length L_{PP} can fall within a range of 2.5 mm to 8.0 mm, or 3.2 mm to 6.9 mm, or 3.9 mm to 5.7 mm, or can be about 4.8 mm.

In various embodiments, the length L_{PP} of the plug portion 1703 is greater than the length L_{TP} of the tip portion 1702. In some scenarios, having a plug portion 1703 that is longer than the tip portion 1702 can provide increased support to the distal tip 1700, increasing the strength of the connection to the shaft 104. In some embodiments, the ratio of the lengths L_{PP}/L_{TP} can be greater than or equal to 2.0, 3.1, 4.2, or 5.3. In some embodiments, the ratio of the lengths L_{PP}/L_{TP} can be less than or equal to 8.0, 7.1, 6.2, or 5.3. In some embodiments, the ratio of the lengths L_{PP}/L_{TP} can fall within a range of 2.0 to 8.0, or 3.1 to 7.1, or 4.2 to 6.2, or can be about 5.3.

In various embodiments a proximal end of the tip portion 1702 has a larger diameter than a distal end of the plug portion 1703. The distal tip 1700 can further include a tip shoulder 1712 between the tip portion 1702 and the plug portion 1703. In various embodiments, a distal end of the return tube 326 is configured to abut the distal tip 1700 at the tip shoulder 1712 when the distal tip is inserted into the shaft 104.

In various embodiments the plug portion 1703 can include a base section 1707, a ramp section 1704, and neck section 1709. The base section 1707 is configured to be proximal to the neck section 1709 when the distal tip 1700 is inserted into a cryoablation shaft 104.

In various embodiments the base section 1707 can have a length L_{B}. In some embodiments, the length L_{B} can be greater than or equal to 2.0 mm, 2.5 mm, 3.1 mm, or 3.6 mm. In some embodiments, the length L_{B} can be less than or equal to 6.0 mm, 5.2 mm, 4.4 mm, or 3.6 mm. In some embodiments, the length L_{B} can fall within a range of 2.0 mm to 6.0 mm, or 2.5 mm to 5.2 mm, or 3.1 mm to 4.4 mm, or can be about 3.6 mm.

In various embodiments, the base section 1707 has a maximum base diameter D_{B}, max. Along the length of the base section 1707, the diameter of the base section 1707 is not larger than the maximum base diameter. In various embodiments, the maximum base diameter D_{B, min} is approximately equal to the inner diameter of the return tube 326. Alternatively, the maximum base diameter D_{B, max} can be marginally smaller or marginally larger than the inner diameter of the return tube 326. In some embodiments, the maximum base diameter D_{B, max} can be greater than or equal to 0.8 mm, 1.0 mm, 1.2 mm, or 1.3 mm. In some embodiments, the maximum base diameter D_{B, max} can be less than or equal to 2.5 mm, 2.1 mm, 1.8 mm, or 1.3 mm. In some embodiments, the maximum base diameter D_{B, max} can fall within a range of 0.8 mm to 2.5 mm, or 1.0 mm to 2.1 mm, or 1.2 mm to 1.8 mm, or can be about 1.3 mm.

In various embodiments, the neck section 1709 can have a length LN. In some embodiments, the length can be greater than or equal to 0.5 mm, 0.7 mm, 0.8 mm, or 1.0 mm. In some embodiments, the length can be less than or equal to 2.0 mm, 1.7 mm, 1.3 mm, or 1.0 mm. In some embodiments, the length can fall within a range of 0.5 mm to 2.0 mm, or 0.7 mm to 1.7 mm, or 0.8 mm to 1.3 mm, or can be about 1.0 mm.

In various embodiments, the neck section 1709 has a maximum neck diameter D_{N, max}long the length of the neck section 1709, the neck section has a diameter not larger than the maximum neck diameter. In various embodiments, maximum neck diameter D_{N, max} is smaller than the maximum base diameter D_{B, max}. In various embodiments, maximum neck diameter D_{N, max} is smaller than the inner diameter of the return tube 326. In some embodiments, the maximum neck diameter D_{N, max} can be greater than or equal to 0.5 mm, 0.7 mm, 0.9 mm, or 1.1 mm. In some embodiments, the maximum neck diameter D_{N, max} can be less than or equal to 2.0 mm, 1.7 mm, 1.4 mm, or 1.1 mm. In some embodiments, the maximum neck diameter D_{N, max} can fall within a range of 0.5 mm to 2.0 mm, or 0.7 mm to 1.7 mm, or 0.9 mm to 1.4 mm, or can be about 1.1 mm.

In various embodiments the plug portion 1703 can include ramp section 1704 between the base section 1707 and the neck section 1709. In various embodiments the diameter of the plug portion 1703 increases over the length of the ramp section 1704 moving in a proximal direction from the neck section 1709 to the base section 1707. In the example of FIG. 17, the diameter of the ramp section 1704 is substantially equal to the maximum base diameter D_{B, max} at the proximal end of the ramp section and the diameter of the ramp section 1704 is substantially equal to the maximum neck diameter D_{N, max} the distal end of the ramp section. In various embodiments ramp section 1704 is configured to provide a gradual decrease in diameter between the base section 1707 and the neck section 1709. Providing a smooth transition in diameter can prevent the compressive wrap from tearing or being damaged as it is compressed to the shaft.

In various embodiments, the ramp section 1704 can have a length LR. The length LR can vary as a function of the difference between the maximum base diameter D_{B, max} and maximum neck diameter D_{N, max}. For instance, the length LR can increase as the difference between the maximum base diameter D_{B, max} and maximum neck diameter D_{N, max} increases. In some embodiments, the length LR can be greater than or equal to 0.10 mm, 0.15 mm, 0.20 mm, or 0.25 mm. In some embodiments, the length LR can be less than or equal to 1.00 mm, 0.75 mm, 0.50 mm, or 0.25 mm. In some embodiments, the length LR can fall within a range of 0.10 mm to 1.00 mm, or 0.15 mm to 0.75 mm, or 0.20 mm to 0.50 mm, or can be about 0.25 mm.

In various embodiments, the proximal end of the distal tip 1700 comprises one or more curved edges 1706. In various embodiments, the curved edges 1706 are configured to prevent the distal tip 1700 from damaging the shaft 104 as the shaft navigates turns and corners during a cryoablation procedure.

Referring now to FIG. 18, a perspective view of a distal end of a cryoablation shaft sealed with distal tip is shown in accordance with various embodiments herein. In various embodiments, the distal tip 1700 is configured to seal a distal end of the return tube 328 of a cryoablation shaft 104.

In various embodiments, the return tube 326 can be sealingly joined to the distal tip 1700 by any suitable method or methods. To form the connection between the return tube 326 and the distal tip 1700, as seen in FIG. 18, the distal tip 1700 can then be inserted into the return tube 326. A compressive element such as a compressive wrap 1808, or the like can then be wrapped around the distal end of the return tube 326. The compressive wrap 1808 can be wound tightly around the shaft 104, compressing the return tube 326 between the compressive wrap 1808 and distal tip 1700. Such a process forms a leak tight seal between the return tube 326 and the distal tip 1700.

In various embodiments, a compressive element, such a compressive wrap 1808 is configured to surround the return tube 326 and the plug portion 1703 of the distal tip 1700, sealing the distal tip to the return tube. In various embodiments, the compressive wrap 1808 is configured to apply a consistent compressive force around the return tube 326, sealing the distal tip 1700 to the return tube 326. In various embodiments, the compressive wrap has a minimum tensile strength. For example, . . . . In various embodiments, the compressive wrap is stable in its properties, including stable in tensile strength and dimensionally stable, at the range of temperatures encountered during a cryoablation procedure. In various embodiments, the compressive wrap has a low creep relaxation over time. In various embodiments, the compressive wrap is biocompatible.

In various embodiments, the compressive wrap can be a monofilament, a multi-filament material, a unifilar material, a multi-filar material, a metal, a plastic, or a composite material. In various embodiments, the compressive wrap is configured to allow termination of the wrap while maintaining tension.

In various embodiments, the compressive wrap 1808 includes a filament 1809 wrapped around the return tube 326 and the plug portion 1703 of the distal tip 1700. In various embodiments, the filament 1809 is configured to be wrapped around the return tube 326 and the plug portion 1703 of the distal tip 1700 with a wrap threshold tension. For instance, the filament 1809 should be able to withstand hanging an object of predetermined mass without breaking. The predetermined mass can be 0.5 kg, 1 kg, 1.5 kg, or greater. In some embodiments, the filament 1809 is hung with the predetermined mass during the wrapping process to apply sufficient wrap tension.

In various embodiments, the compressive wrap can have a tensile strength. In some embodiments, the tensile strength can be greater than or equal to 1.0 gigapascal (GPa), 1.8 GPa, 2.7 GPa, or 3.5 GPa. In some embodiments, the tensile strength can be less than or equal to 6.0 GPa, 5.2 GPa, 4.3 GPa, or 3.5 GPa. In some embodiments, the tensile strength can fall within a range of 1.0 GPa to 6.0 GPa, or 1.8 GPa to 5.2 GPa, or 2.7 GPa to 4.3 GPa, or can be about 3.5 GPa.

In various embodiments, the filament 1809 can be formed from any suitable material or materials including, but not limited to fibrous materials such as monofilament suture, including monofilament suture made from high density polyethylene, stainless steel wire, Tungsten wire, multi-stranded sutures, or the like.

In various embodiments, the filament 1809 can be formed from a material or materials configured to transmit electrical signals (e.g., conductive wire). In such an embodiment, one or more sensors can be placed at the distal end of the shaft 104 and the sensor data can be conveyed from the sensor(s) to the console via the electrically conductive filament. In one example, an electromagnetic position sensor can be included at the distal tip 1700 of the shaft 104 and is configured to aid the physician in positioning the shaft within the patient's anatomy. In such a configuration, the compressive wrap 1808 can be configured to carry a low voltage that would be translated into a position from the distal tip 1700 to the console. In another example, the compressive wrap 1808 could incorporate fiber optic cable(s) configured to provide information regarding the shape of the target anatomy. Any other suitable sensor or combination of sensors can be included at the distal end of the shaft including but not limited to temperature sensors, pressure sensors, strain sensors, or the like.

In various embodiments, the filament 1809 can be formed from one or more high resistance wires. The high resistance wires can be configured to enable electrical heating of the expansion chamber 106 of the shaft 104. The electrical heating can facilitate more rapid thawing of ice balls, which can decrease cryoablation procedure time and reduce the likelihood of tract seeding, where harmful tissue adheres to the shaft be distributed to other portions of a patient's anatomy.

In various embodiments, the filament 1809 is wound around the return tube 326. In various embodiments, the filament 1809 can have a closed pitch where each wind of the filament is touching an adjacent wind. Alternatively, the filament 1809 may have a pitch that is greater than or equal to 0.5 mm, 1.0 mm, 1.5 mm, or 2.0 mm, or can be an amount falling within a range between any of the foregoing. Alternatively, the filament 1809 may be a plurality of discrete pieces attached along the return tube 326.

In the example of FIG. 18, the compressive wrap 1808 contains two layers of filament 1809 wound along the length of the return tube 326. In some configurations, having multiple layers of filament 1809 in the compressive wrap 1808 can enhance the uniformity of the compression along the length of the return tube 326. In alternate configurations, the compressive wrap 1808 can contain one or three or more layers of filament wound along the length of the return tube 326.

In some various embodiments, the diameter of the filament 1809 can vary depending on the material of the filament and its ability to hold tension during manufacturing. In some embodiments, the diameter of the filament 1809 can be greater than or equal to 0.002 mm, 0.004 mm, or 0.005 mm. In some embodiments, the diameter of the filament 1809 can be less than or equal to 0.007 mm, 0.006 mm, or 0.005 mm. In some embodiments, the diameter of the filament 1809 can fall within a range of 0.002 mm to 0.007 mm, or 0.004 mm to 0.006 mm, or can be about 0.005 mm.

In various embodiments, the compressive wrap 1808 can include an adhesive 1810 configured to encapsulate the filament 1809. The adhesive 1810 is configured to maintain the wrap threshold tension of the filament 1809 and can enhance the material properties of the compressive wrap 1808 such as biocompatibility, radiopacity, or the like. Alternatively, the adhesive 1810 can be applied to discrete portions of filament 1809 (such as at the ends). Examples of suitable adhesive or adhesives that can be used to encapsulate the filament 1809 include cyanoacrylate adhesive and LOCTITE® adhesive available from Henkel Adhesive Technologies, which has a location in Bridgewater, New Jersey, USA, or the like. In various embodiments, the compressive wrap 1808 can have a wrap length L_{CW}. In some embodiments, the length can be greater than or equal to 1 mm, 2 mm, 3 mm, or 4 mm. In some embodiments, the length can be less than or equal to 7 mm, 6 mm, 5 mm, or 4 mm. In some embodiments, the length can fall within a range of 1 mm to 7 mm, or 2 mm to 6 mm, or 3 mm to 5 mm, or can be about 4 mm.

In various embodiments, the distal tip 1700 is configured to be inserted into the distal end of a shaft 104 up to plane 1705. When the distal tip is inserted into the shaft 104, a distal end of the return tube 326 is configured to abut the distal tip 1700 at the tip shoulder 1712. In alternate configurations, a distal end of the return tube 326 is configured to abut the distal tip 1700 proximally to the tip shoulder 1712. In either configuration, the tip shoulder 1712 is configured to prevent the distal tip 1700 from slipping into the return tube 326, in the event that the distal tip starts to slip with respect to the return tube.

When the distal tip 1700 is inserted into the shaft 104, a distal end of compressive wrap 1808 is configured to abut the distal tip 1700 at the tip shoulder 1712. In alternate configurations the distal end of the compressive wrap 1808 is configured to abut the distal tip 1700 proximally to the to the tip shoulder 1712. In either configuration, the tip shoulder 1712 is configured to prevent the compressive wrap 1808 from slipping off of the distal tip 1700 in the event that the compressive wrap starts to slip with respect to the distal tip. In the embodiment of FIG. 18, both the compressive wrap 1808 and the return tube 326 terminate at tip shoulder 1712.

In various embodiments, the compressive wrap 1808 compresses the return tube 326 to the distal tip 1700 along the ramp section 1704 of the distal tip. In various embodiments, the gradual change in distal tip diameter over the ramp section 1704 is configured to prevent damage to the return tube 326 when the return tube is compressed to the distal tip 1700 with compressive wrap 1808.

Near the center of the distal end, the tip portion 1702 forms a plateau portion 1701 having a planar surface in some embodiments. In some embodiments, the plateau portion 1701 is a remnant of a step in the manufacturing process which provides support to the distal tip 1700 during the compressive wrapping process. In some embodiments, the distal tip 1700 includes a removeable extension section extending away from plateau portion 1701. The removeable extension section is removed, such as by cutting, after the compressive wrapping process is completed, which may leave a plateau portion 1701.

During some embodiments of the manufacturing process, the removeable extension section is inserted into a rotating chuck and the rotating chuck is clamped down on the removable extension section. The chuck is rotated while the compressive wrap is applied, thereby rotating the entire distal tip 1700 during the process of applying the compressive wrap. The rotating chuck supports the distal tip during the process of applying the compressive wrap. After the compressive wrap is applied, the removeable extension process is removed, such as by cutting.

In other embodiments, the plateau portion 1701 is not present, even when the removeable extension section is used during the manufacturing process, such as if a smooth curved tip remains after the removeable extension section is cut.

In various embodiments, the tip portion 1702 can have a first tip diameter D_{T1} corresponding to the tip diameter at plane 1705. In some embodiments, the first tip diameter D_{T1} can be greater than or equal to 1 mm, 1.1 mm, 1.2 mm, 1.3 mm, or 1.4 mm. In some embodiments, the first tip diameter D_{T1} can be less than or equal to 1.8 mm, 1.7 mm, 1.6 mm, 1.5 mm, or 1.4 mm. In some embodiments, first tip diameter D_{T1} can fall within a range of 1.0 mm to 1.8 mm, or 1.1 mm to 1.7 mm, or 1.2 mm to 1.6 mm, or 1.3 mm to 1.5 mm, or can be about 1.4 mm. In various embodiments, the first tip diameter D_{T1} is approximately equal to the outer diameter of the return tube 326. Such a configuration creates a smooth transition between the return tube 326 and the distal tip 1700.

### Markers for Imaging Systems

The joint 334 (FIG. 3) and the distal tip 108 (FIGS. 8 and 10) are configured to appear on imaging systems. The joint 334 can include a marker band of radiopaque materials, such as tungsten or platinum iridium or other appropriate materials. The material and dimensions of the distal tip 108 can be configured to appear on imaging systems. Examples of materials for the distal tip 108 include stainless steel and nitinol. The markers at either end of the active zone of the device assist the physician with visually identifying the location of the active zone of cryoablation with respect to the patient's anatomy and the target zone for cryoablation. Examples of imaging systems include ultrasound, fluoroscopy, cone beam CT, and MRI systems.

### Introduction Options

In various embodiment, the catheter system is delivered using a sheath introduction system. An example of a sheath introduction system is a steerable sheath. Alternatively, the catheter system can be steerable. In another embodiment, the catheter system includes a monorail lumen along a portion of the catheter to facilitate

### INTRODUCTION

The concepts described herein can be applied in the context of and used in connection with cryoablation systems and components described in the following four U.S. nonprovisional patent applications, which are filed on the even date herewith, which are incorporated by reference herein in their entireties: U.S. Nonprovisional patent application Ser. No. ______, titled “Cryoablation Catheter Shaft Construction,” having attorney docket number 115.0421USU1; U.S. Nonprovisional patent application Ser. No. ______, titled “Safety Devices for Cryoablation Probe,” having attorney docket number 115.0422USU1; U.S. Nonprovisional patent application Ser. No. ______, titled “Multiple Gas Circuit Connector and Method for Cryoablation System,” having attorney docket number 115.0423USU1; and U.S. Nonprovisional patent application Ser. No. ______, titled “Delivery Systems for Cryoablation Device,” having attorney docket number 115.0424USU1.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cryoablation probe comprising:
a working fluid circuit;
a vacuum circuit; and
a shaft, the shaft comprising:
a supply tube;
a return tube surrounding the supply tube;
an insulated portion, wherein the vacuum circuit runs through the insulated portion between the return tube and an insulating shaft; and
an expansion chamber extending distally to the insulated portion, wherein fluid from the working fluid circuit travels through the supply tube and expands in the expansion chamber;
a distal tip configured to seal a distal end of the return tube, the distal tip comprising:
a tip portion extending distally from the distal end of the return tube;
a plug portion configured to be inserted inside of the return tube; and
a compressive element surrounding the return tube and the plug portion of the distal tip, configured to seal the distal tip to the return tube.

2. The cryoablation probe of claim 1, wherein at least a portion of the plug portion has a first portion that has a diameter that is larger than an inner diameter of the return tube and wherein the compressive element is a ring.

3. The cryoablation probe of claim 2, the plug portion comprising a neck section having a minimum neck diameter and a base section having a minimum base diameter, wherein the base section is located proximally to the neck section along the return tube, wherein the minimum neck diameter is smaller than the inner diameter of the return tube, and wherein the base section includes the first portion having a diameter larger than the inner diameter of the return tube.

4. The cryoablation probe of claim 3, the plug portion comprising a first shoulder between the neck section and the base section.

5. The cryoablation probe of claim 3, the neck section comprising a protrusion configured to form a seal with the return tube, wherein the ring surrounds the protrusion.

6. The cryoablation probe of claim 3, the base section comprising a barb configured to form an interference fit with the return tube, wherein the first portion of the plug portion is the barb.

7. The cryoablation probe of claim 1, wherein a proximal end of the tip portion has a larger diameter than a distal end of the plug portion, and wherein the distal tip comprises a tip shoulder between the tip portion and the plug portion, wherein a distal end of the return tube abuts the distal tip at the tip shoulder.

8. The cryoablation probe of claim 7, wherein a distal end of the compressive element surrounds the distal tip at the tip shoulder or distally to the tip shoulder.

9. The cryoablation probe of claim 2, wherein the ring comprises a swage ring.

10. The cryoablation probe of claim 2, wherein the ring comprises a metal ring.

11. The cryoablation probe of claim 1, wherein the compressive element is a compressive wrap surrounding the return tube and the plug portion of the distal tip, configured to seal the distal tip to the return tube.

12. The cryoablation probe of claim 11, the plug portion comprising a neck section having a neck diameter and a base section having a base diameter, wherein the base section is located proximally to the neck section along the return tube, wherein the neck diameter is smaller than an inner diameter of the return tube, and wherein the base diameter is equal to or larger than the inner diameter of the return tube.

13. The cryoablation probe of claim 12, the plug portion comprising a ramp between the neck section and the base section, wherein a diameter of the plug portion increases over a length of the ramp moving in a proximal direction from the neck section to the base section.

14. The cryoablation probe of claim 12, the compressive wrap comprising a filament wrapped around the return tube and plug portion of the distal tip with a wrap threshold tension.

15. The cryoablation probe of claim 14, further comprising an adhesive configured to encapsulate the compressive wrap, wherein the adhesive is configured to maintain the wrap threshold tension of the filament.

16. The cryoablation probe of claim 1, the tip portion comprising an atraumatic surface.

17. The cryoablation probe of claim 1, wherein a proximal end of the distal tip comprises one or more curved edges.

18. A method of making a shaft component of a cryoablation probe, comprising:
providing a tube component of a cryoablation expansion chamber;
providing a distal tip configured to seal a distal end of the tube component, the distal tip comprising:
a tip portion configured to extend distally from the distal end of the tube component; and
a plug portion configured to be inserted inside of the tube; and
inserting the distal tip plug portion into a distal end of the tube component so that the plug portion is within the tube component and the tip portion extends distally from the distal end of the tube component;
applying a compressive force to a compressive element surrounding the tube component and the plug portion of the distal tip, wherein the compressive element seals the distal tip to the tube component; and
positioning a supply tube within the tube component, wherein the supply tube is configured to supply a fluid of a working fluid circuit, wherein the fluid expands in the expansion chamber.

19. The method of claim 18, wherein the compressive element is a ring and applying the compressive force comprises:
positioning the ring surrounding the tube component and the plug portion of the distal tip; and
compressing the ring;
wherein at least a first portion of the plug portion has a diameter that is equal to or larger than an inner diameter of the tube component.

20. The method of claim 18, wherein the compressive element is a compressive wrap and wherein applying the compressive force comprises wrapping the compressive wrap around the tube component and the plug portion of the distal tip.
